(19) United States
(12) Patent Application Publication
(10) Pub. No.: US 2010/0280395 A1
(43) Pub. Date: Nov. 4, 2010

(54) System and Method for Hypertension Management

(75) Inventor: Kin-Yuan Lin, Taipei (TW)

Correspondence Address: SHOEMAKER AND MATTARE, LTD, 10 POST OFFICE ROAD - SUITE 100, SILVER SPRING, MD, 20910, US

(73) Assignee: MICROLIFE INTELLECTUAL PROPERTY GMBH, Widnau (CH)

(21) Appl. No.: 12/161,406

(22) PCT Filed: Jan. 19, 2007
(86) PCT No.: PCT/EP07/50533

§ 371 Date: Jul. 18, 2008

(30) Foreign Application Priority Data
Jan. 20, 2006 (EP) 06100653.2

(51) Int. Cl. A61B 5/021

(52) U.S. Cl. 600/485

(57) ABSTRACT

A system (1) for hypertension management comprises at least one home blood pressure measuring device (10) for blood pressure measurements of an individual at the individual's home. Furthermore, the system (1) comprises at least one point of care measuring system (20) with a point of care blood pressure measuring device (21). This allows blood pressure measurements at a point or care. The system is provided with data communication means for transferring data from the home blood pressure measuring device (10) to a web server (35) or the point of care measuring system (20).

The invention relates to a system and a method for hypertension management. Hypertension is a major cause for severe diseases in most industrialised countries, and it is growing significantly in developing and poor countries. In view of treatment of hypertension it is thus important to provide reliable blood measurement results of individuals. In traditional patient care processes, patients, physicians and pharmacists are different, isolated entities. Patient's data or medical records are not circulated or shared among these parties. More importantly, methods and procedures for patients to take blood measurements at home and also methods for point of care measurements are not standardised. Consequently, information needed for diagnosis, treatment or medication adjustment for hypertension may be inaccurate. There have been no incentives to physicians or pharmacists to use standardised guidelines for diagnosis, treatment or for providing sufficient consultations and education materials or services. The overall control rate for hypertension management therefore is only 21%-27% (see e.g. Judy Possidente Kaufman, etc., “The role of Home Blood Pressure Monitoring in Hypertension Control”, Journal of Clinical Hypertension 3(3): 171-173, 2001). Current hypertension management systems thus are not successful.

There are current proposals to start pay for performance programs (P4P) for physicians in view of reimbursements regarding treatment of hypertension. Similarly, medication therapy management (MTM) programs are currently planned in view of reimbursements for pharmacists in order to make sure drug compliance. In these programs, specific documentation is required as evidence to prove that physicians and pharmacists have provided necessary services, follow-up and keeping track of hypertension treatment outcomes.

It is an object to overcome the drawbacks of what is known, in particular to provide a system and a method for hypertension management which allows achieving a high overall control rate. Furthermore, the system and method according to the present invention shall provide reliable blood pressure measurements upon which a treatment of an individual can be based. It is a further object of the invention to provide a method and a system for hypertension management which easily allows physicians or pharmacists to comply with requirements of quality standard programs such as P4P or MTM programs.

According to the present invention, these and other objects are solved with a system and a method according to the independent patent claims.

The system according to the invention is primarily used for hypertension management. The system includes at least one home blood pressure measuring device for blood pressure measurement at an individual's home. Of course, depending on the number of individuals treated with such a system, there can be a plurality of such home blood pressure measuring devices. The system further comprises at least one point of care measuring system. The point of care measuring system includes a point of care blood pressure measuring device for blood pressure measurement of an individual at a point of care. Typically, a point of care in this context is a physician's office, a pharmacy or any other kind of centralised health care institution. According to the invention, the system is provided with data communication means for transferring data from the home blood pressure measuring device to a central web database accessible by a care person such as a physician or a pharmacist or for transferring data to the point of care measuring system. The system allows to combine blood pressure measurements made at the individual's home and measurements done by a physician or a pharmacist at a point of care. While it is known that home measurements may be the best alternative for accurate blood pressure measurements (see e.g. Laurie Barclay, “Home measurement best for accurate BP monitoring”, BMJ.2002; 325:254-257 or Judy Possidente Kaufman et al., “The role of home blood pressure monitoring in hypertension control”, J Clin Hypertens 3(3): 171-173, 2001), it is generally accepted that physicians or pharmacists should in addition make their own blood pressure measurements for verification purposes. According to the system of the present invention, a patient may take a plurality of home blood pressure measurements. Data of these measurements can be made accessible to care persons, physicians, physicians by uploading the data to a database, in particular a web based database or by uploading the data to the point of care measurement system through the data communication means. Direct uploading of the data at the point of care will be particularly preferable for patients or individuals which are not used to utilize computers.

According to a preferred embodiment of the invention, the home blood pressure measuring device is adapted to determine blood pressure in accordance with specific, predetermined measurement criteria. Such measurement criteria based on clinical considerations are known. A device for carrying out such measurements is disclosed in the co-pending PCT application PCT/EP2005/052730, the content of which hereby is incorporated by reference. According to a preferred embodiment of the invention, the home blood pressure monitoring device is provided with an interface for entering a measurement schedule. This measurement schedule can be based on measurement guidelines. In particular and in accordance with a further preferred embodiment, in addition to a prestored schedule based on measurement guidelines, a schedule individually prepared for the patient and based on the judgement of a care person can be entered into the device. In particular, the interface may be formed by the data communication means. Such an interface allows for an easy definition of a custom made measurement schedule individually for each patient, e.g. depending on his daily habits. A care person e.g. a physician may centrally define such a measurement schedule without the need of manually entering data into the device by the care person or the patient.

According to a further embodiment of the invention, the home blood pressure measuring device can be operated in two operating modes: in a first, automatic measurement mode measurements are automatically made in accordance with the measurement schedule. In a second, manual measurement mode, measurements are made only after manual interaction by the user. In particular, the measurement schedule may also define if for a specific predetermined period of time the device shall be operated in the automatic or in the manual measurement mode. In particular, measurements during night or during working time may be automatic whereas measurements in the evening or in the morning may be started manually, depending on the patients individual preferences.

According to still a further preferred embodiment and according to another aspect of the invention, the home blood pressure measuring device is provided with a calculating means which is adapted for the determination of at least one of white coat hypertension or masked hypertension. It has been found (see e.g. P.Verdecchia, “Reference values for ambulatory blood pressure and self-measured blood pressure based on prospective outcome data”: blood pressure monitoring 2001, 6:323-327 or J. M. Mallion, “Detection of masked hypertension by home blood pressure measurements: is the number of measurements an important issue?” Blood pressure monitoring 2004, 9:301-305) that patients in whom clinical blood pressure measurements are normal nevertheless may suffer from too high blood pressure. Three phenotypes of such out of physician's office hypertension have been described, namely morning hypertension, nocturnal hypertension and daytime stress hypertension. According to this aspect of the invention, there is provided an automatic blood pressure measuring device usable by the patient which allows for detection of these three types of hypertension and therefore helps to increase the efficiency of an anti-hypertension strategy. The detection of three types of hypertension by home blood pressure monitoring in combination and with reference to point of care measurements achieves a more effective prevention of cardiovascular events through a perfect 24 hour blood pressure control. Masked hypertension and white coat hypertension effects can be eliminated.

According to a further embodiment of the invention, the system can be operable in a plurality of different measurement modes e.g. in an initial/diagnostic mode, in a treatment mode and in a follow-up mode. According to still another preferred embodiment of the invention, the operation mode can be selected through a data interface, in particular through online or remote interaction by a care person.

Furthermore, the home blood pressure device can operate in a normal so called WatchBP home) mode and in an extended (so called ABPM lite) mode, depending on the requirements of an individual patient. In the WatchBP home mode, morning and evening measurements are triggered manually. In addition to these measurements, in the ABPM lite mode, worksite and night measurements are triggered automatically on a specific number of working days, typically for 1, 2 or 3 working days. The device can be provided with a switch for switching between these measurement modes.

According to still a further embodiment of the invention, the point of care blood pressure measuring device is adapted to determine blood pressure in accordance with specific, predefined measurement guidelines. Such guidelines are e.g. known from Pickering et al. (Pickering et al., Recommendations for blood pressure measurements in humans and experimental animals, Hypertension 2005; 45: 142-161). A point of care blood pressure measuring device appropriate for such measurements is disclosed in the co-pending application having same filing date in the name of same applicant which is incorporated herein by reference.

According to another aspect of the invention, the system includes at least one point of care measuring device for blood pressure measurements at a point of care. The point of care measuring system is designed to operate in accordance with predetermined quality standards, in particular in accordance with the criteria of a pay for performance program or in accordance with the criteria of a medication therapy management program. Such a device easily allows a physician to generate reports necessary to fulfil the requirements of such a program. In particular, the point of care blood pressure measuring device may generate reports necessary for the physician or pharmacist to be reimbursed for his or her services on hypertension management and treatment. While such a system with a capability to generate quality standard reports already has considerable advantages as such, it will be understood that in combination with the above mentioned integrated system having home and point of care measuring devices, additional advantages can be achieved.

According to a further aspect of the invention, the system may further comprise at least one home based medication compliance monitoring device. This device may be designed for data communication with the central database. In addition to trans-mission of blood pressure data, medication compliance data of the patient may be transmitted therewith. In particular, if a point of care system is arranged at the physician's and at the pharmacist's premises, medication compliance and blood pressure data which are relevant for pharmacists or physicians can be accessed to the point of care system.

In another preferred embodiment, the system is provided with a central database. This database particularly may be a web based database. In the database, blood pressure data and drug compliance data may be stored. Thereby, these data are in parallel accessible by a plurality of users such as a physician, a pharmacist, the patient, nurses or other care givers. Knowledge of physicians and pharmacists and measurements done by patients thereby can be integrated. With the system of the present invention, patients, physicians, pharmacists and the BP measurements taken at different sites and patient medication compliance information are not isolated anymore. They can be integrated in one overall system. General information about disease management knowledge can be downloaded on demand if required by a physician or a pharmacist.

Based on such a database it is further possible to generate individual education reports or care plans for each patient. Depending on the patient's condition, in particular depending on blood pressure values for three different phenotypes of hypertension (see below) but also parameters such as age, weight or the like, individual reports and care plans may be generated.

In view of the above, it is particularly preferred if according to the system of the present invention there is provided a point of care system at least one physician's office and at least one pharmacy.

Another aspect of the invention relates to a blood pressure measuring device for blood pressure measurements at a point of care. The automatic blood pressure measurement system is provided with means for generating reports to be used in a quality control program, in particular in a pay for performance or in a medication therapy management program. The blood pressure measuring system typically includes a specific blood pressure measuring device for clinic blood pressure measurements. In addition, this system is provided with software instructions, which allow the generation of reports fulfilling requirements of such pay for performance programs. In particular, a report in the form of a prospective data collection flow sheet as suggested in “Clinical performance measures, Hypertension, Tools developed by physicians for physicians” provided by the American College of Cardiology/American Heart Association/Physician Consortium for Performance Improvement can be automatically generated with the system according to the invention. The reports typically may include, but no limited to, all data records, clinical performance measures, plan of care, etc.

According to a further aspect of the invention, there is provided a method for hypertension management. According to this method, in a first step, blood pressure values of an individual are determined at the individual's home with a home blood pressure measuring device. In a further step, blood pressure values of the individual are determined at a point of care with a point of care blood pressure measuring device. The blood pressure data measured at the individual's home are then transferred from the home blood pressure measuring device to a central database or to a point of care blood pressure measuring system.

It is preferred that blood pressure data determined at the individual's home and/or blood pressure data determined at the point of care are determined in accordance with respective clinical recommendation or measurement criteria.

According to a further aspect of the invention, in a method for hypertension management, in a first step blood pressure values of an individual are determined at a point of care. In a further step, reports used in a quality insurance program such as a pay for performance or a medication therapy management program are automatically generated by the software tool.

According to a further preferred embodiment of the invention, in addition to the blood pressure data, medication compliance data determined in a medication compliance monitoring device at the individual's home are also transferred to a central database or to a point of care blood pressure measuring system.

According to a further preferred embodiment of the method according to the invention, data relating to the patient, further individual data such as blood glucose, weight, age or the like are transferred to the central database, in particular an internet database, which can be accessed from several point of care systems. Furthermore, individual education reports for an individual can be automatically generated from such a system.

The invention will now be explained in more detail with reference to the following embodiments and the accompanying drawings, in which

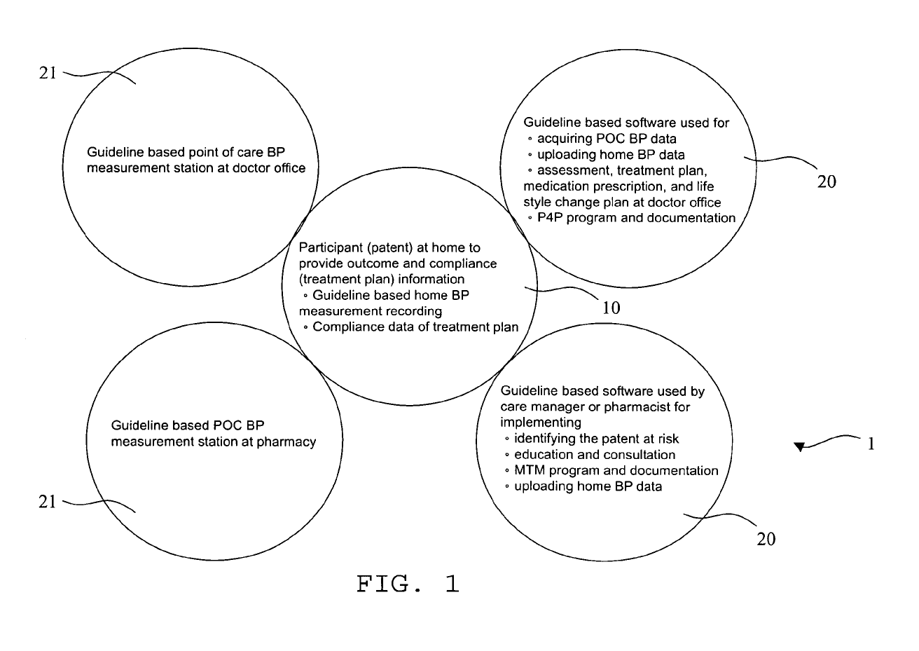

FIG. 1 shows a schematic representation of components of a system according to the present invention

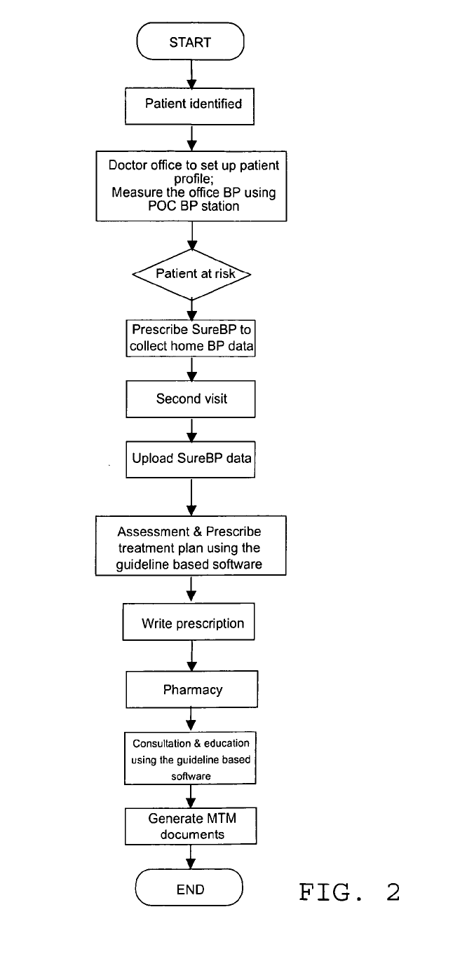

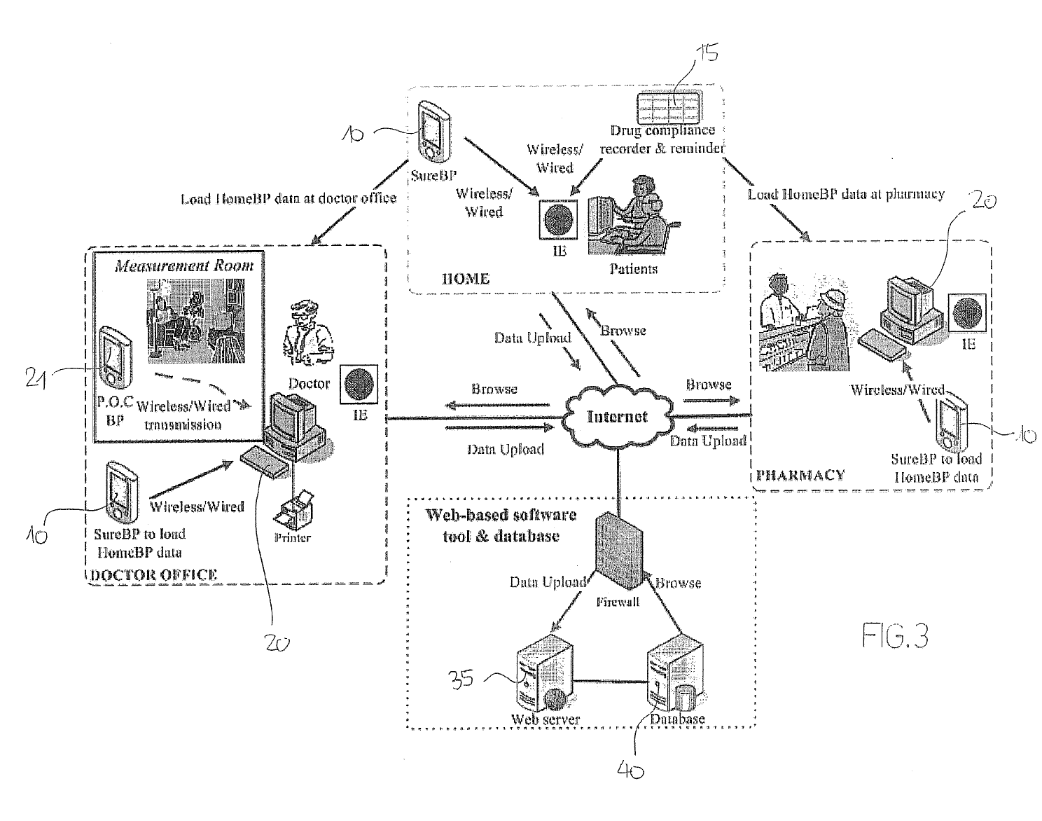

FIG. 2 shows a flow chart with steps of a method according to the present invention

FIG. 3 shows a general overview of a system according to the invention

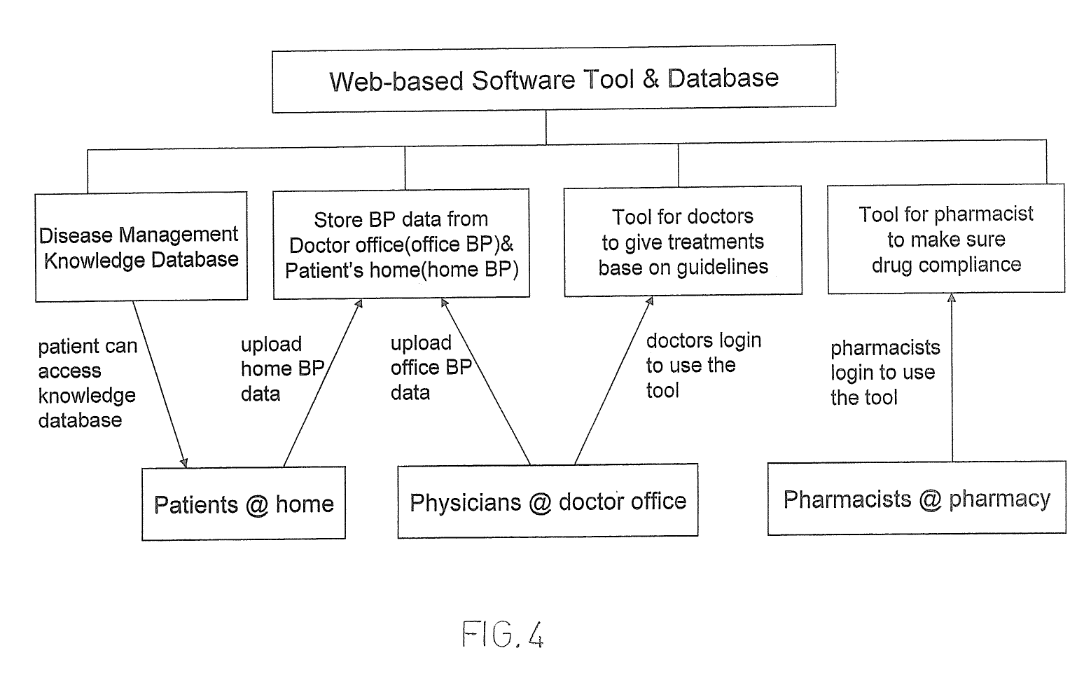

FIG. 4 shows a block diagram of different components and mutual communication of a system according to the invention

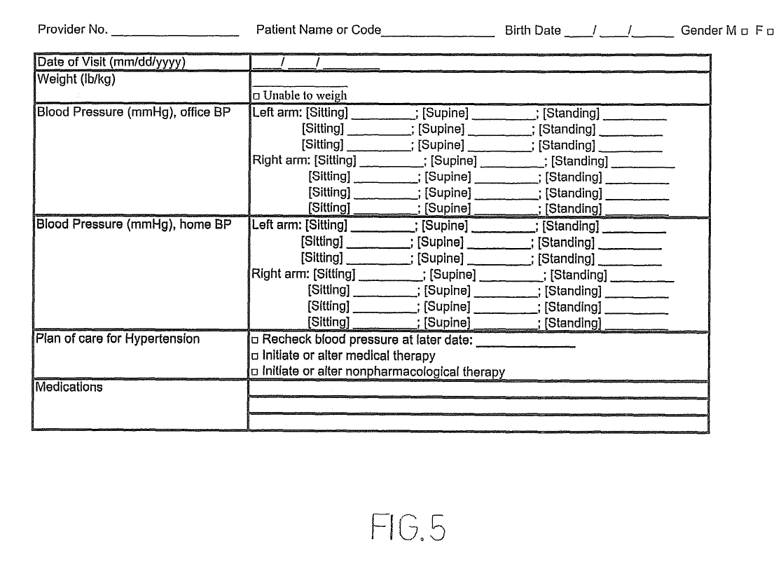

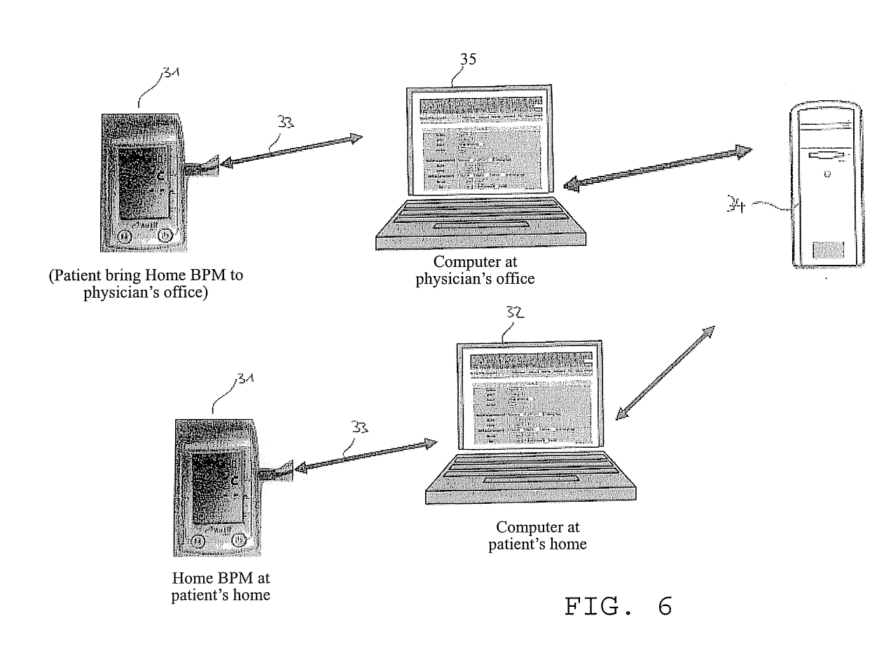

FIG. 5 shows an example of a P4P report.

FIG. 6 schematically shows an alternative, preferred embodiment of a home blood pressure measuring device

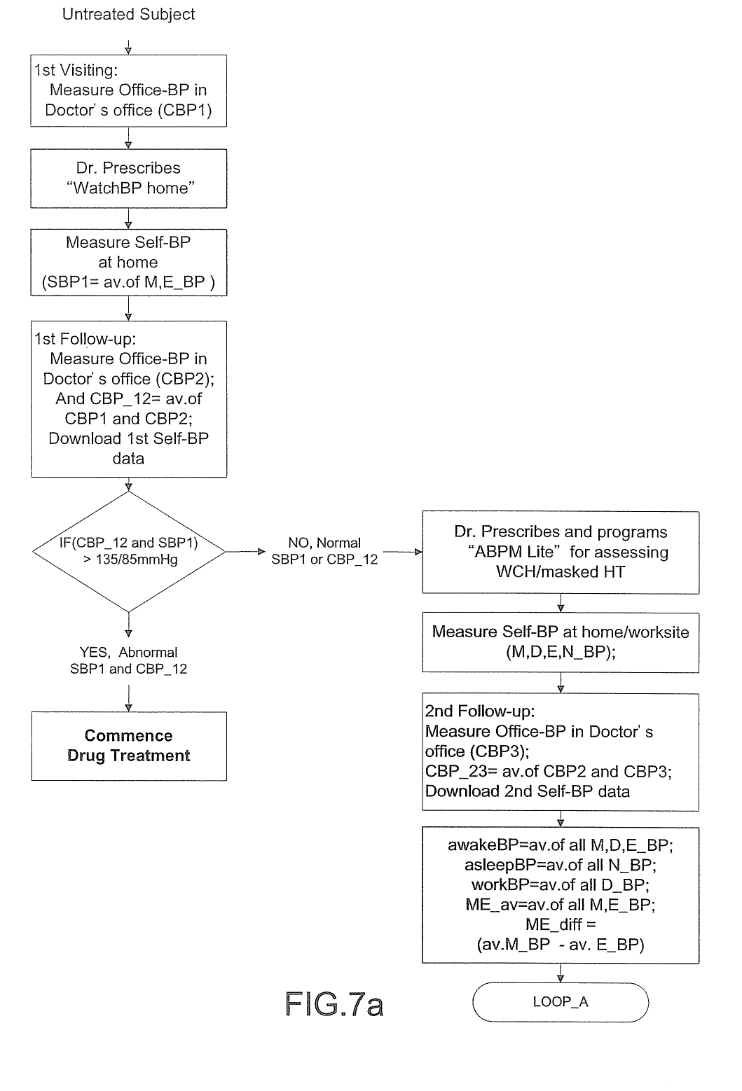

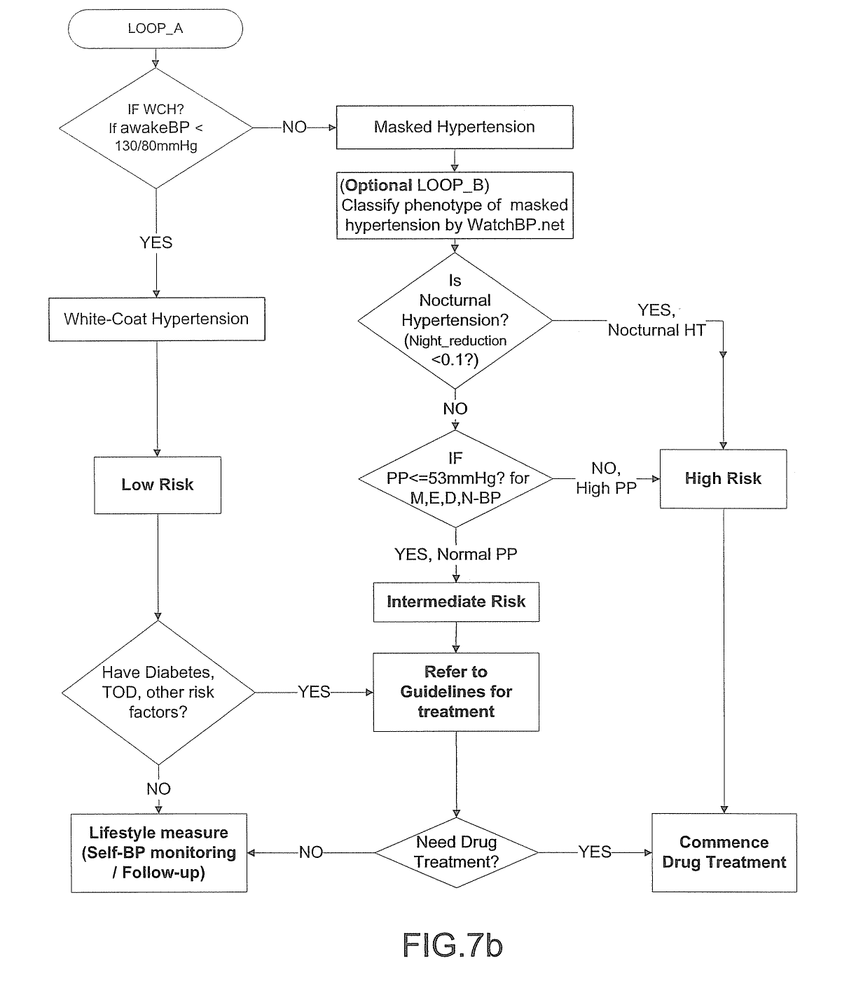

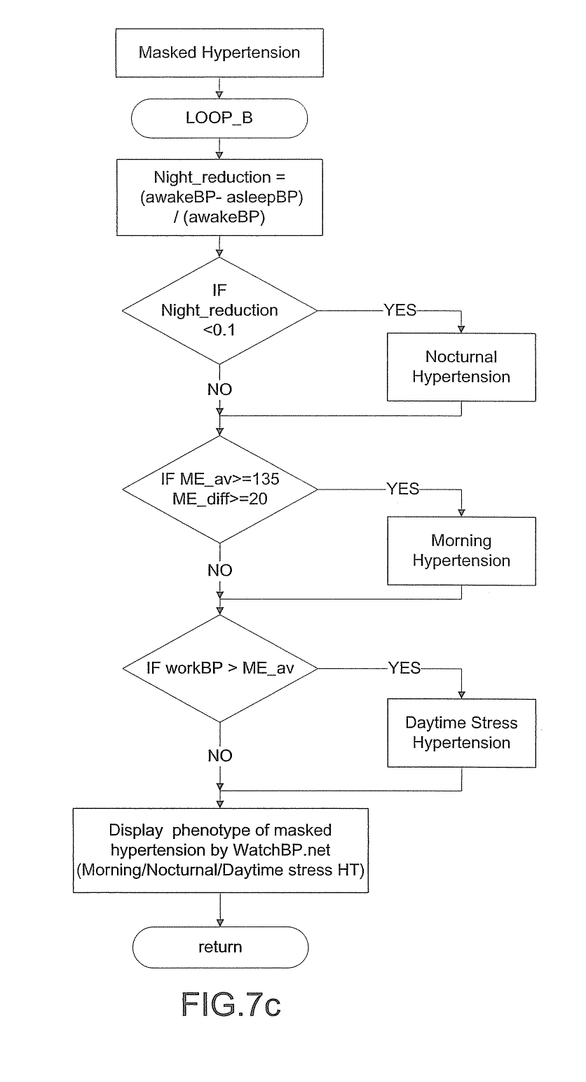

FIGS. 7a, 7b, 7c show a flow chart of operation of a system for detection of white coat and masked hypertension

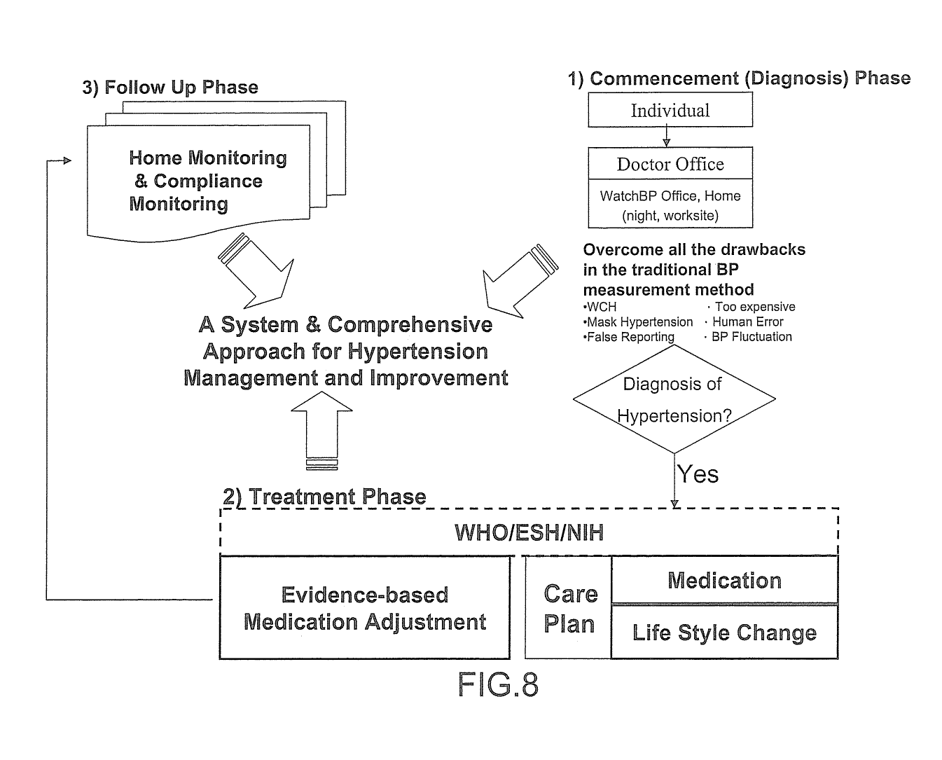

FIG. 8 shows a schematic overview of a systematic and comprehensive approach for hypertension management with a system in accordance with the present invention

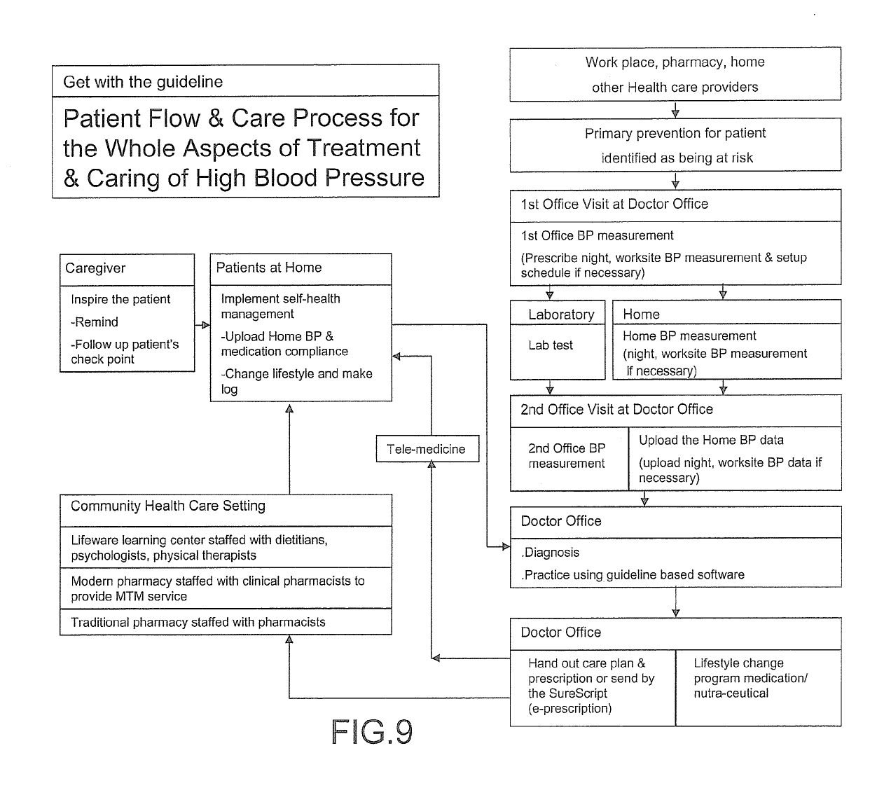

FIG. 9 schematically shows the care process in accordance with the present invention

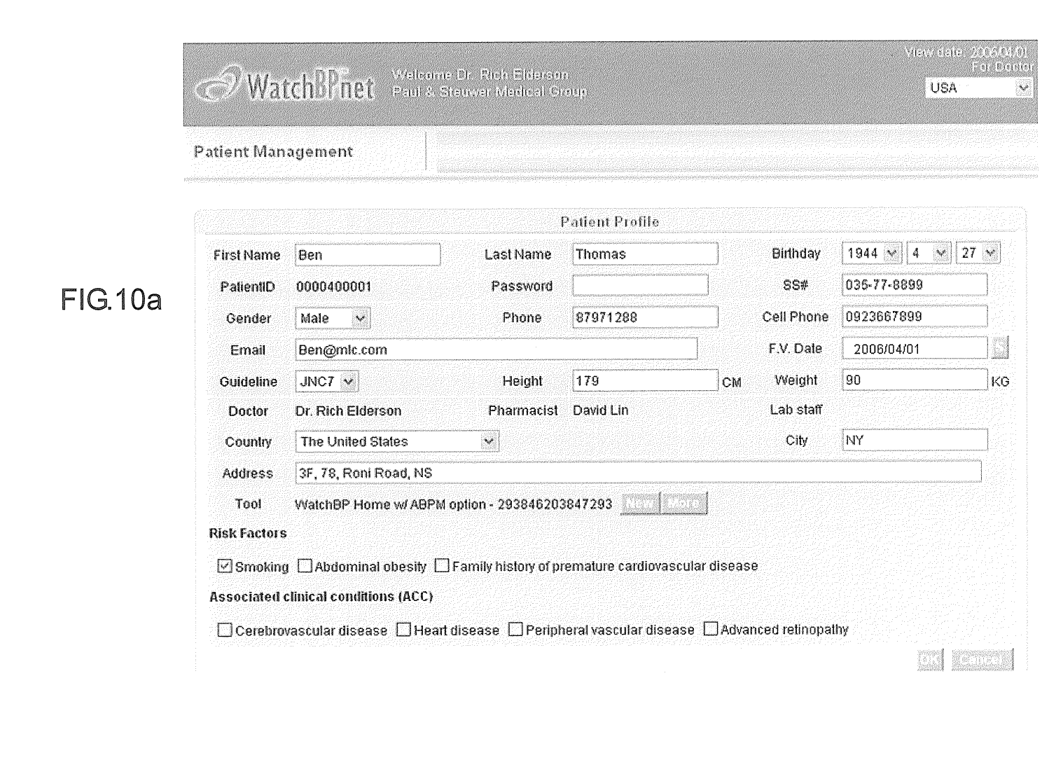

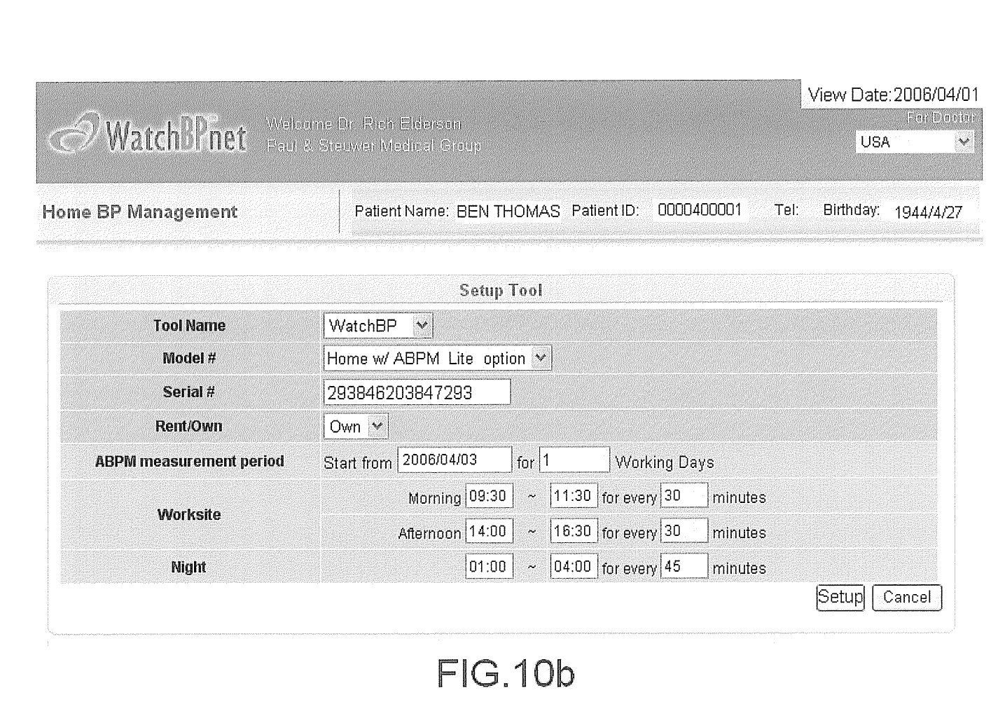

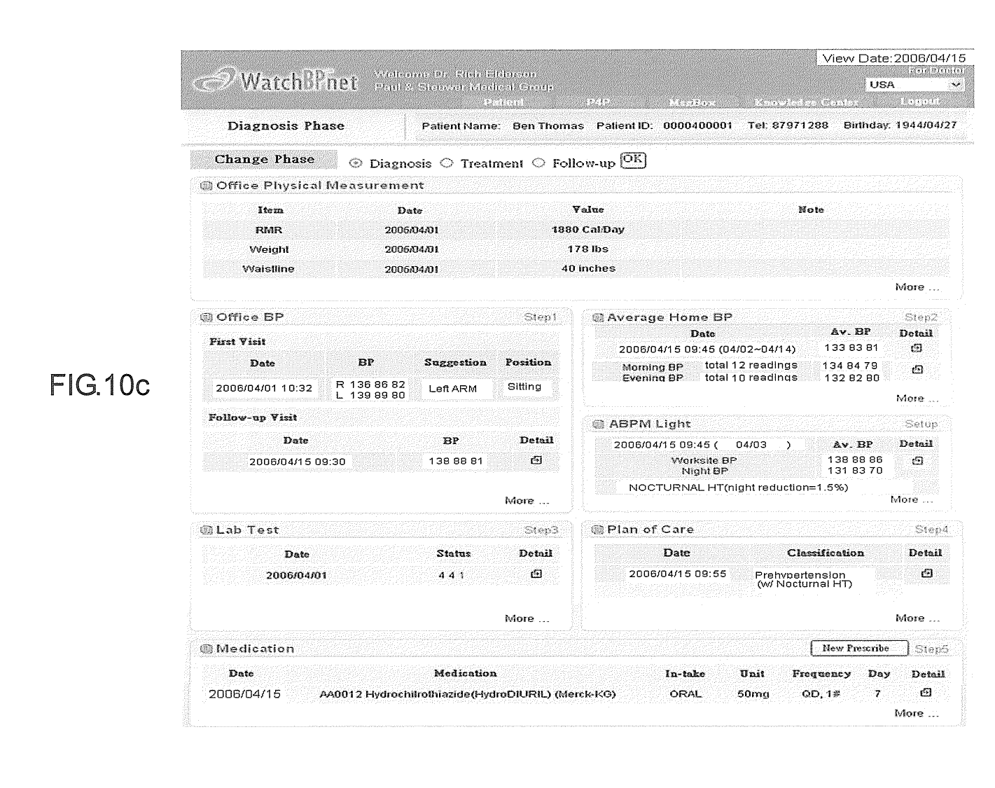

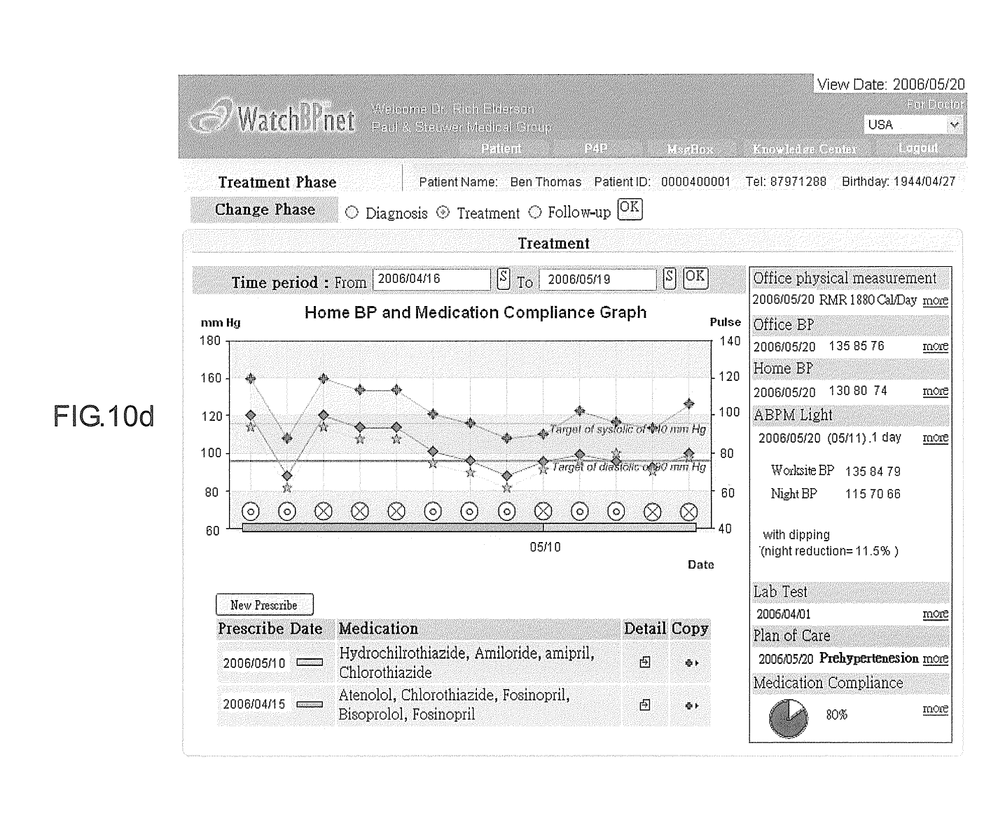

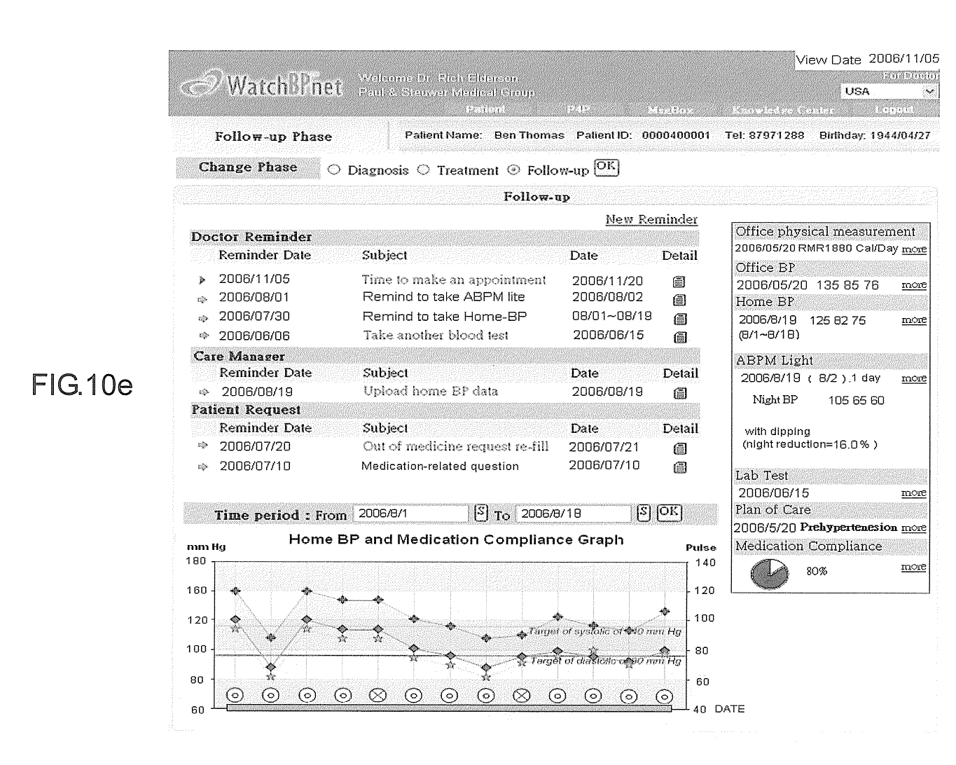

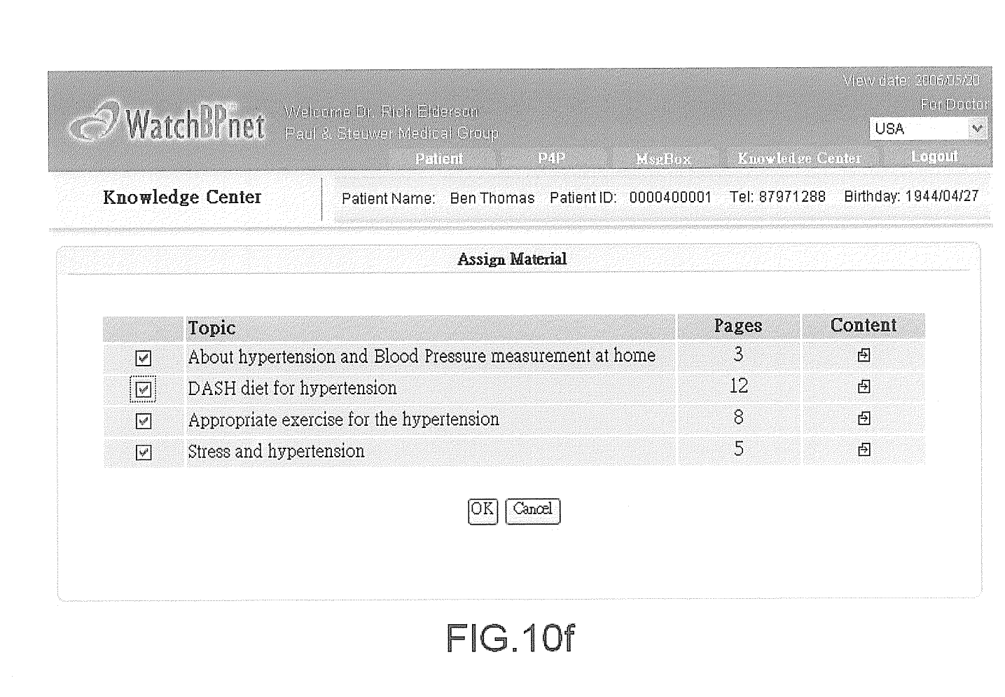

FIGS. 10a to 10f show screen shots of a web interface for operation of a system according to the present invention

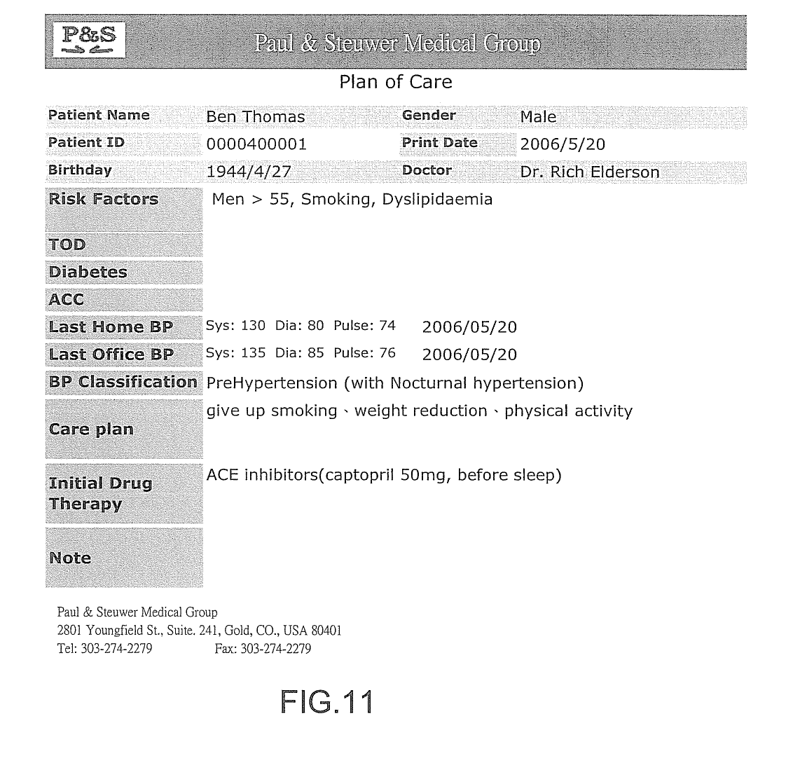

FIG. 11 shows an example of a plan of care generated in accordance with the present invention.

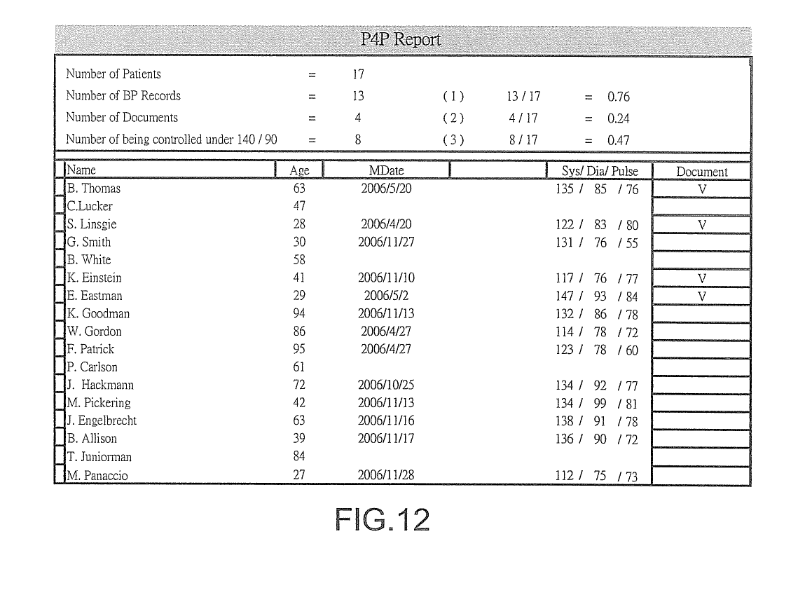

FIG. 12 shows an example of a P4P report generated by a specific care person.

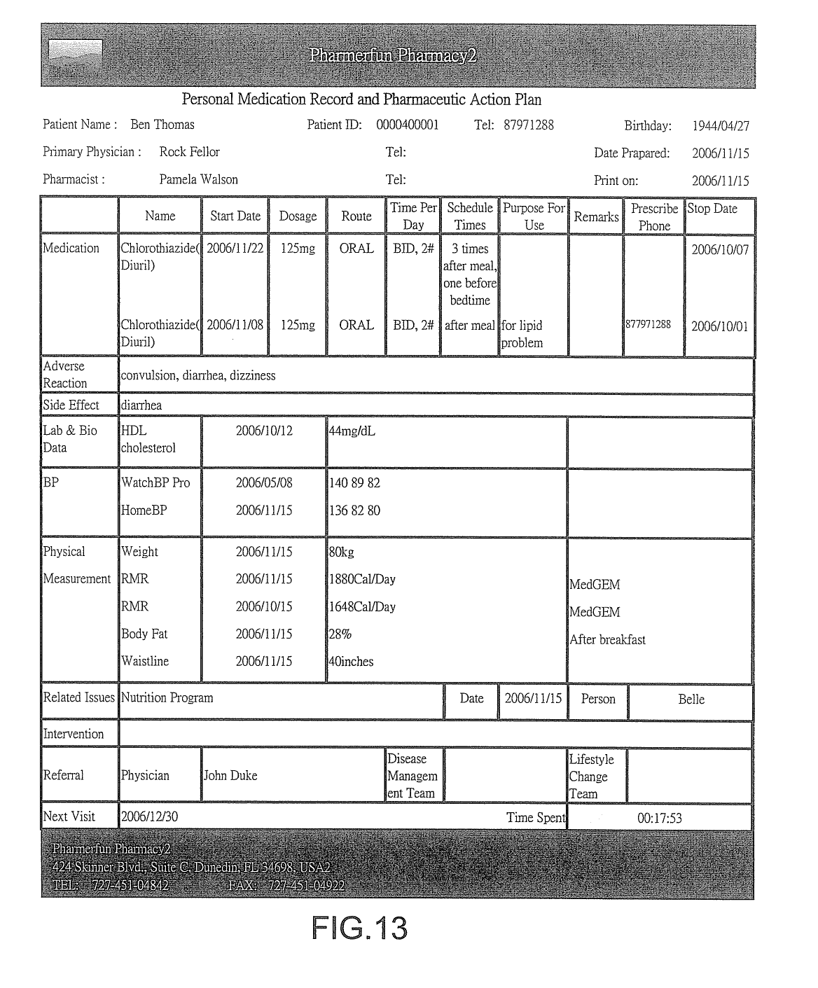

FIG. 13 shows an embodiment of a personal medication record for a specific patient and

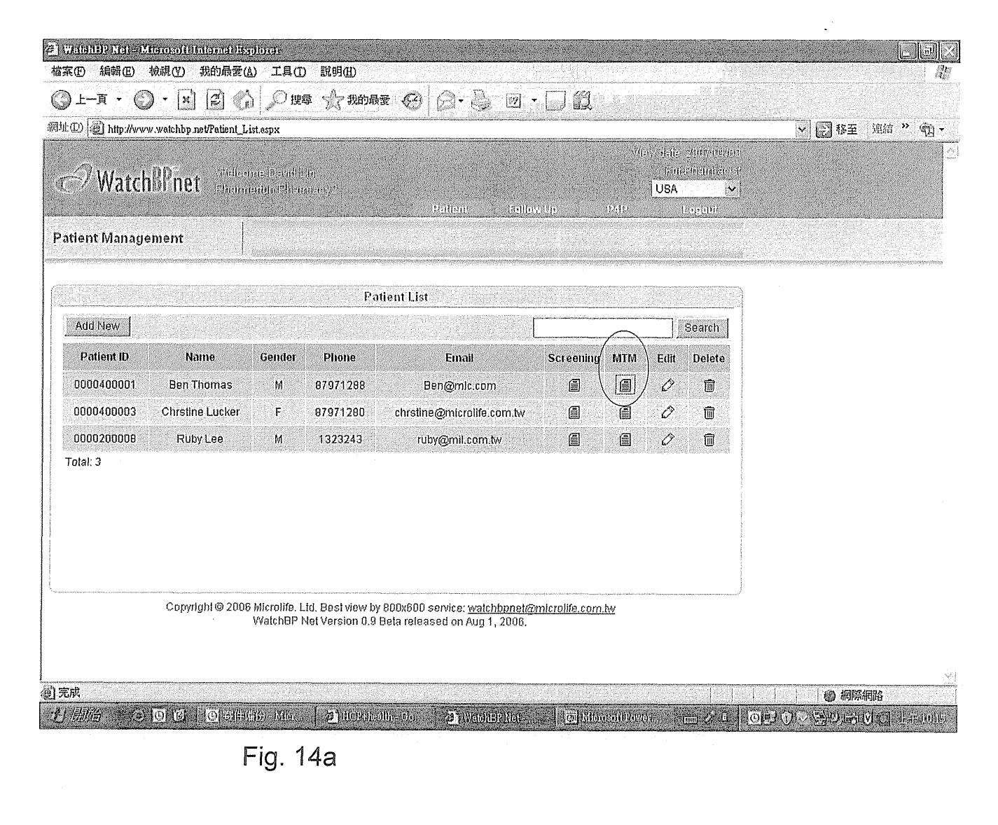

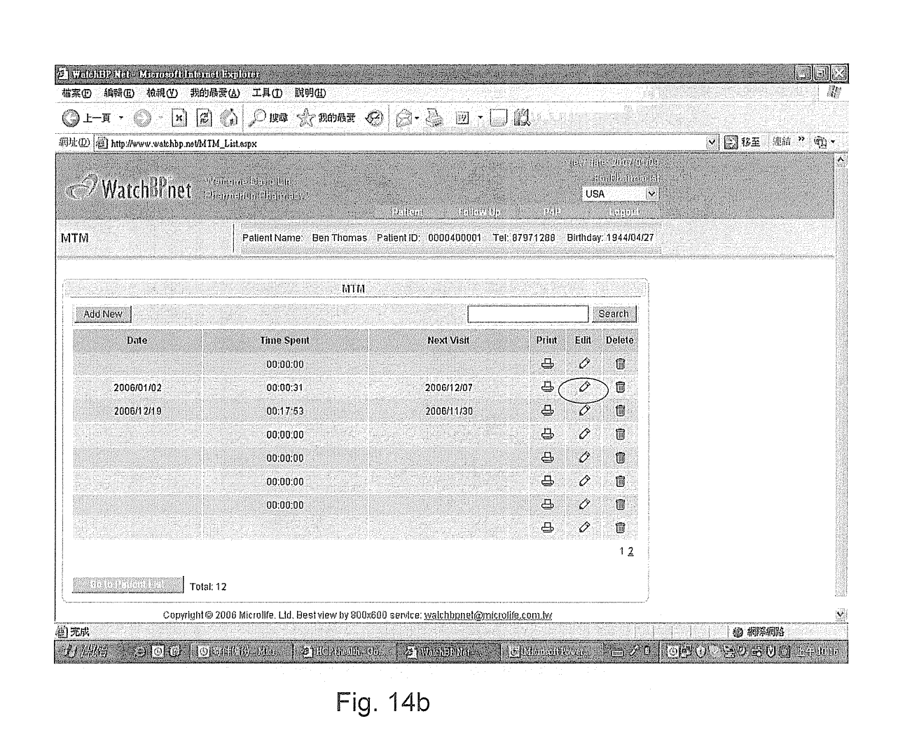

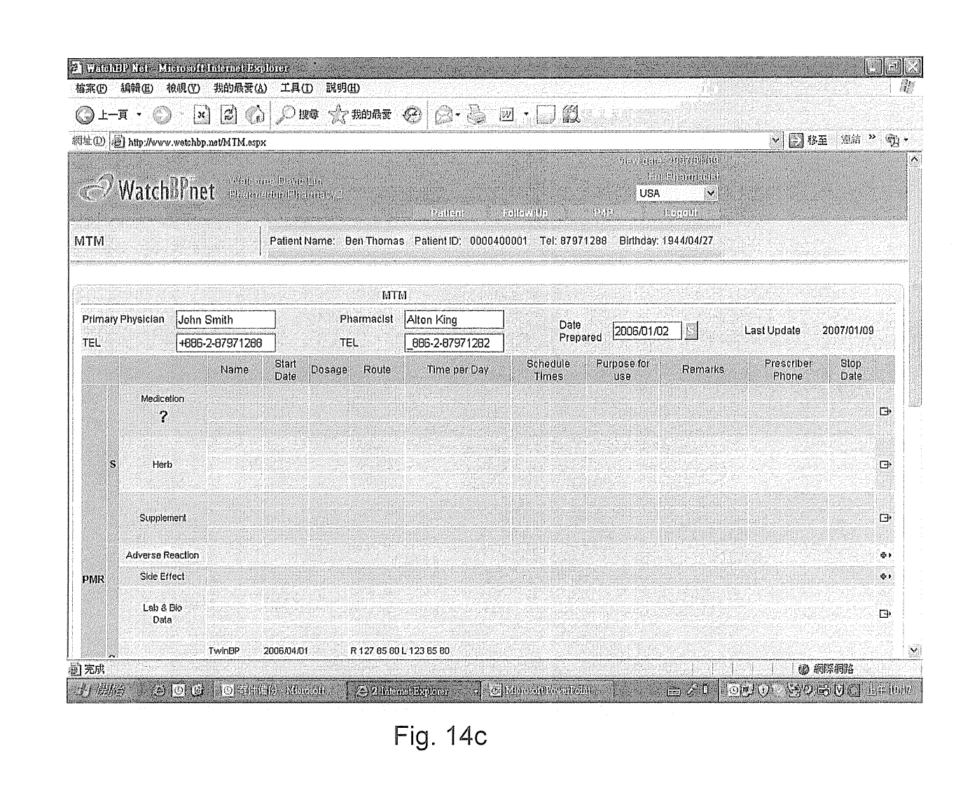

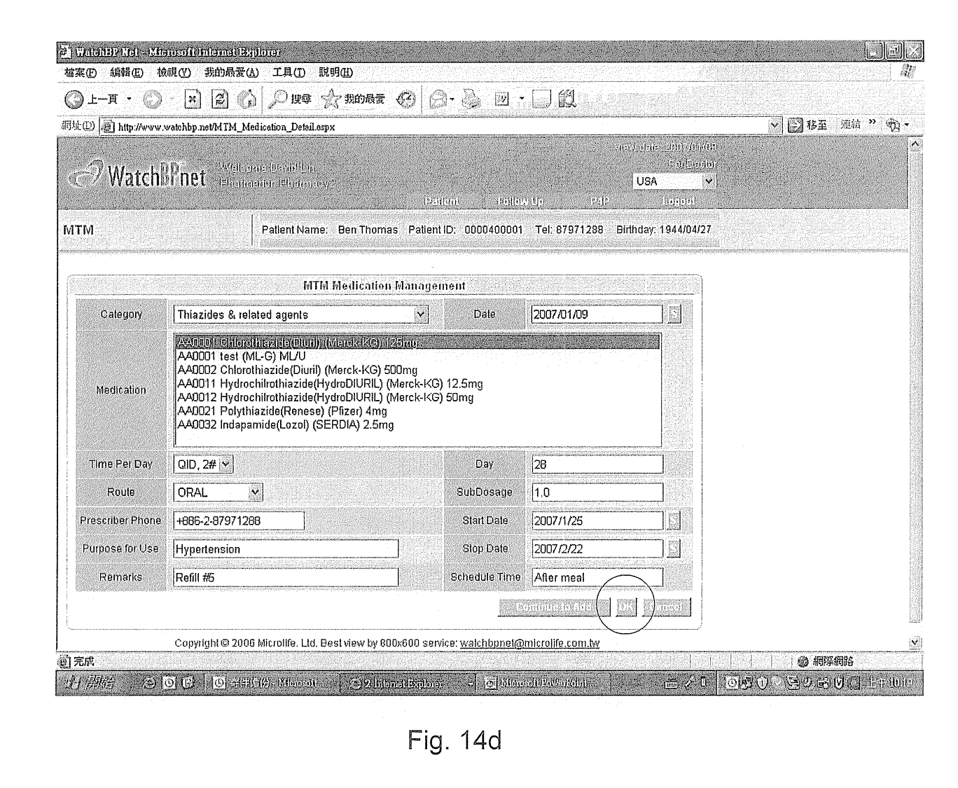

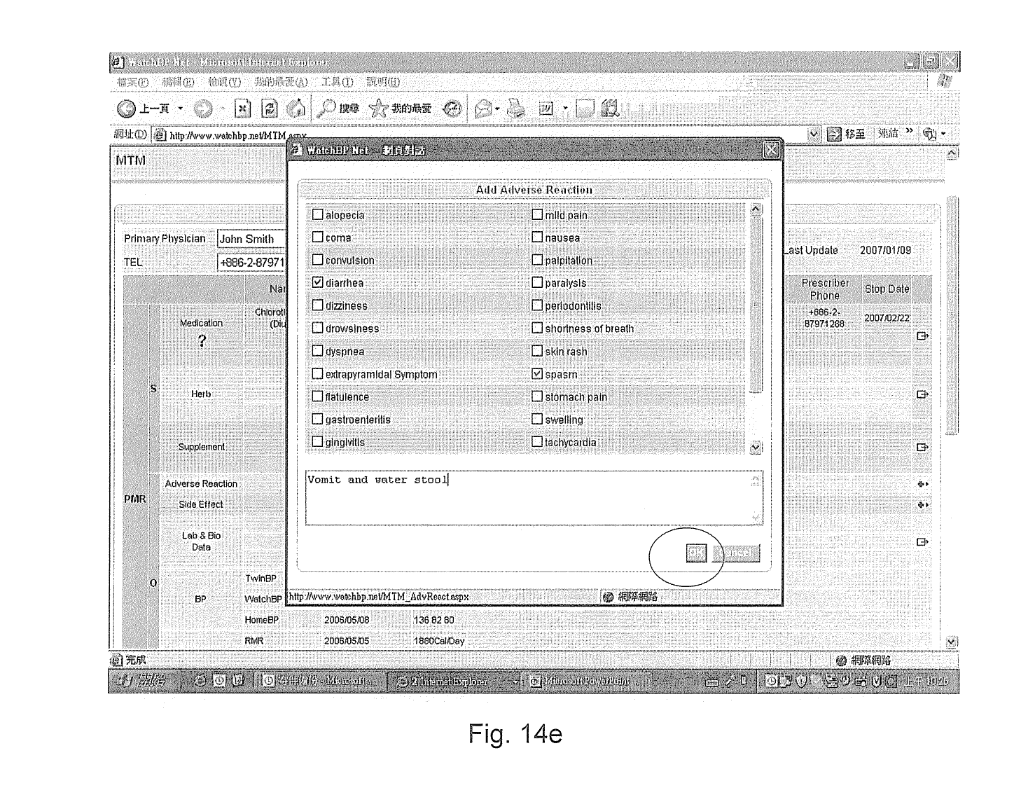

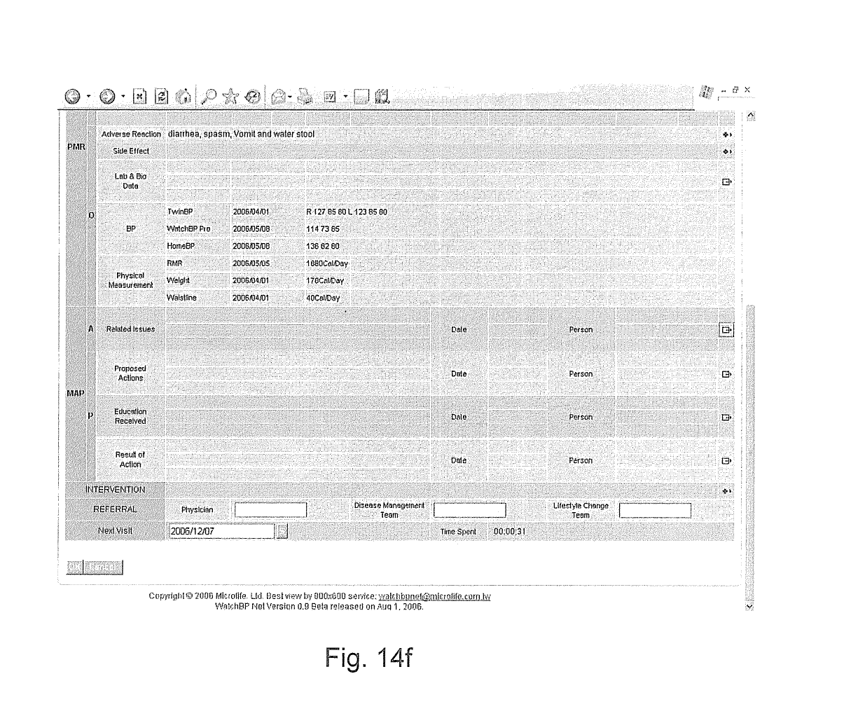

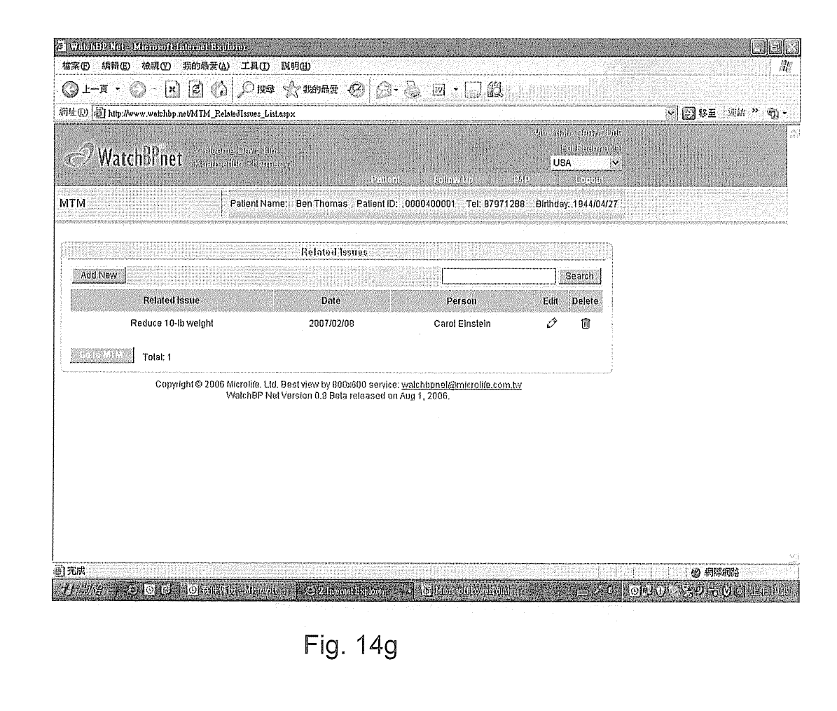

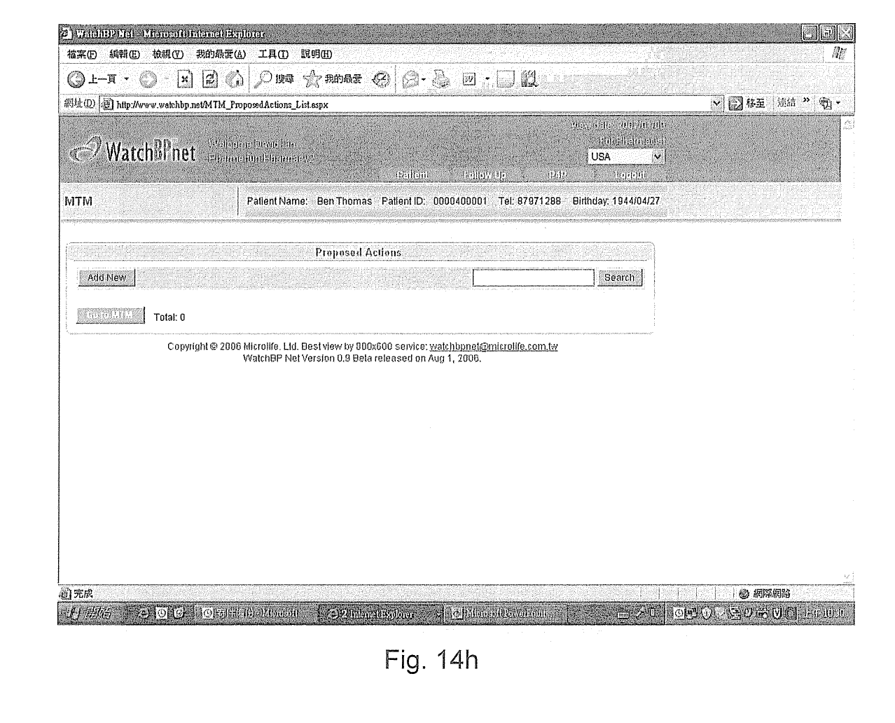

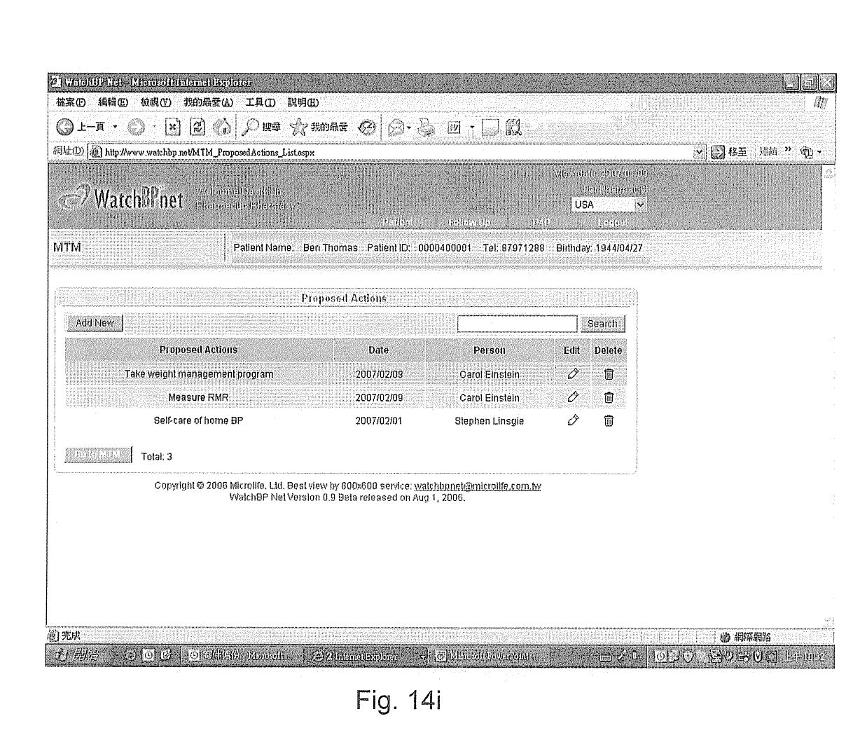

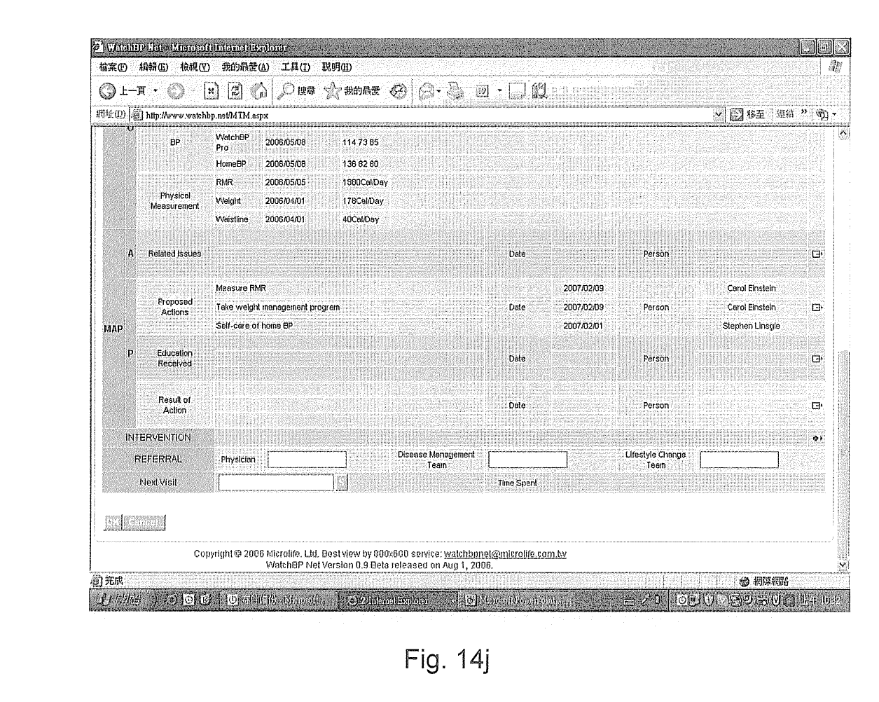

FIGS. 14a to 14j show typical screen shots of web browser pages directed to a patient medication records in medication therapy management.

FIG. 1 schematically shows several components of a system 1 for hypertension management in accordance with the present invention. A first component includes a home blood pressure measuring device 10. An individual or patient is carrying out blood pressure measurements with the home blood pressure measuring device 10 in accordance to specific guidelines or a physician schedule prescribed by a physician or another care person. In addition, compliance data for treatment plan are recorded by a drug compliance recorder device at the patient's home (see FIG. 3).

A first point of care measuring system 20 is located at a physician's office. The point of care measuring system 20 includes guideline based software used for several purposes. The software could also be on a web server for physicians to be used online, e.g. by logging in through the internet (see FIG. 3). The software is used for acquiring blood pressure data at the point of care according to specific guidelines. The software is furthermore used to upload data which have been measured by the individual at the individual's home with the home blood pressure measuring device 10. Included in the software is furthermore information relating to assessment, prescription, treatment plans or lifestyle change plans. Finally, the software used in the system is capable of generating reports and documentation in response to a pay for performance program.

A second point of care measuring system 20 is used at a pharmacy. The system includes guideline based software used by a care manager or a pharmacist for implementing different tasks. The software could be locally at pharmacist's computer or on the web server 35 for pharmacists for online use. One of the task is to identify whether a specific individual or patient is at risk. Another task of the software is to provide education and consultation information. Furthermore, the software is adapted to provide reports in answer to requirements of an MTM program and appropriate documentation. Finally, the software is also used for uploading data from a home blood pressure measuring device 10 which data have been acquired individually by the patient. With a point of care blood pressure measuring device 21 point of care blood pressure measurements may be made at a physician's office or at a pharmacy.

FIG. 2 shows a flow chart of operation of the method according the invention carried out at a physician's office. In a first step after start of the procedure a patient is identified. In the physician's office, a patient profile is set up. With the point of care blood pressure measurement device 21 the individual's blood pressure is measured. If the patient is not at risk, the method according to the invention is stopped. If the patient is found to be at risk to suffer from hypertension, he is prescribed with a home blood pressure measuring device 10 (called SureBP herein after) to collect home blood pressure data. The home blood pressure measuring SureBP 10 is operating in accordance with predefined measurement criteria. In particular, the home blood pressure measuring device 10 is programmed so as to automatically remind the patient to take measurements at appropriate times.

In a second visit, the patient or individual uploads the blood pressure data measured at home with the home blood pressure measuring device 10. This may be e.g. done by connecting the home blood pressure measuring device 10 through an interface to the point of care measuring system 20. Upload of the data can also be made online e.g. by uploading the data to an internet database.

In a further step, an assessment of the patient is made and an appropriate treatment plan is prescribed. For prescription of the treatment plan, a guideline based software is used. Typically, the software includes guidelines such as recommended by WHO/ESH/NIH. Based on the assessment and the treatment plan, a prescription is written and the individual is referred to a pharmacy. At the pharmacy, consultation and education of the individual is made on the basis of a guideline based software running at the pharmacy. Furthermore, the point of care system 20 at the pharmacy generates MTM documents necessary for the pharmacist to be reimbursed.

The communication interface for uploading data from the home blood pressure measuring device to the point of care measuring system e.g. may be a USB connection. Any other kind of connection may be conceivable.

By transferring data to an internet based database, trends, medication compliance or other information may be presented to the physician, pharmacist or to other care givers.

Medication compliance may be made with a programmed medication schedule device. On a screen, reminders for taking medication may be provided. Confirmation of compliance may be entered into the device. The medication schedule can be synchronised online.

FIG. 3 shows a general overview of the system according to the present invention. Mutual communication between the different components are shown. At the patient's home, data are transferred from a drug compliance recorder 15 and reminder and/or from a home blood pressure measuring device 10 to a web database 40 through the internet. Through the internet, contents of this database also may be browsed. Blood pressure data also are stored in the home blood pressure measuring device 10, so that they can be transmitted to a point of care measuring system 20 if the device 10 is brought to the point of care. Data can be uploaded from this point of care measuring system 20 to the central database 40 through the internet so that they will be accessible for all the parties involved. Software may be made available on a web server 35.

A point of care system 20 is arranged each at a physician's office and at a pharmacy. Data gathered at these locations, in particular data measured by a point of care blood pressure measuring device 21 or prescription or consultation data may be uploaded to the database 40 through the internet. Furthermore, data contained in the database 40 can be accessed from the pharmacy or from the physician's office through the internet. In particular, relevant measurement data but also knowledge information can be accessed.

FIG. 4 shows in more detail contents of the web-based software tools and database and interactions thereof with different users. In particular, a patient can access a disease management knowledge database from home and the patient can upload home blood pressure data measured with the home blood pressure measuring device 10 to the central web database where they are stored. The physician at the physician's office can also upload blood pressure data which have been gathered with the point of care measuring device 21 to the database 40. In addition, a physician can access specific tools in view of treatments based on guidelines. Pharmacists can access to tools in order to ensure drug compliance. Furthermore, patients can upload compliance data to the web database 40.

FIG. 5 shows an embodiment of a P4P report which will be completed by means of the present invention.

FIG. 6 schematically shows an alternative embodiment of a home blood pressure device 31. The home blood pressure measuring device 31 is connected to a personal computer or a laptop 32 by means of an USB connection 33. Instead of a USB connection, any other kind of data communication connection known to those skilled in the art could be used. The laptop 32 is connected to a web-based database server 34, e.g. by an internet connection. The measurement schedule stored in the home blood pressure measuring device 31 which is based on predetermined measurement guidelines may be embodied in the device or may be amended through the USB connection 33. In particular, a care person such as a physician may enter a measurement schedule individually for a patient and based on measurement guidelines through a physician's office laptop computer 35 and USB connection 33 or by an internet browser. Through the connection between the web database 34, the laptop 32 and the home blood pressure measuring device 31, such measurement schedule may be transferred to the home blood pressure measuring device 31. Consequently, there is no need for the care person or for the patient to individually enter a specific measurement schedule into the device 31.

In order to enhance the results of the system for hypertension management, the system, i.e. the home blood pressure measuring device and/or the point of care measuring system may be provided with a calculating arrangement for determination of masked hypertension, nocturnal hypertension or white coat hypertension. Because of the integration of a home blood pressure measuring device and a point of care measuring device information necessary for determination of white coat, nocturnal or masked hypertension are available in one single system so that automatic determination is possible. FIGS. 7a, 7b and 7c schematically show flow charts for the operation of such a system.

In FIG. 7a there is shown a flow chart of the steps which are carried out for a patient possibly suffering from hypertension consulting a physician in accordance with the present invention. During a first visit, a blood pressure measurement is made in the physician's office with the point of care measuring device. This measurement leads to a measurement result CBP1. This measurement can be made with a professional device hereinafter called “WatchBP Pro”. It is possible to make measurements on both arms in order to determine a preferred measurement arm for subsequent measurements in accordance with known guidelines. Subsequently, the physician prescribes a home blood pressure measuring device, hereinafter referred to as “WatchBP home”. The patient measures the blood pressure at home in accordance with a measurement schedule prescribed by the physician. The patient is asked to measure the blood pressure in the morning and in the evening leading to blood pressure values M_BP and E_BP. An average SBP1 is formed on the basis of this morning and evening measurements. Typically, measurements are repeated for a number of days prescribed by the physician, e.g. for 7 working days. The average SBP1 is formed as the average of all measurements taken during these days. However, the first days reading can be excluded in accordance with suggested home blood pressure measurement guidelines. During this measurement period, the WatchBP home is operating manually and not based on a prescribed measurement schedule yet.

Upon a first follow-up visit at the physician's office, a second measurement is made with the point of care measuring device leading to a measurement result CBP2. An average CBP 12, is formed on the basis the two point of care measurements CBP1 and CBP2. In a further step, the first self measurement blood pressure data acquired by the individual are downloaded to the system. In this context, this system is particularly a web based database server which is accessible from the physician's office.

If the average of the point of care measurement CBP 12 and the average of the home measurements SBP1 are above 135/85 mmHG, it is judged that the blood pressure is abnormally high and a drug treatment is started.

If either the home measured blood pressure or the point of care measurement is normal, the physician prescribes the patient with an ambulatory blood pressure monitor in order to assess whether the patient probably suffers from white coat or masked hypertension. For this purpose, a home blood pressure measuring device is programmed through the connection as described above such as to work in a “ABPM Lite” mode.

In the WatchBP home mode, measurements are made in the morning_and in the evening. These measurements are triggered manually. Typically, on each working day, two subsequent measurements are made. After a first measurement, a countdown of 60 seconds is made and a second measurement is automatically started. The blood pressure reading is formed as an average of these two readings. Measurements are made by default between 6.00 and 9.00 pm. and 6.00 am and 9.00 am. The averages may be calculated and/or stored in the home measurement device.

Optionally, when the device is programmed in the ABPM lite function, worksite and night measurements are made. Typically, one measurement is made during 1, 2, or 3 working days. Measurements are triggered automatically each 15 to 20 minutes during daytime between 9.30 am and 11.30 am and 2.00 pm and 4.30 pm. During the night, measurements are automatically triggered every 45 minutes between 1.00 am and 4.00 am.

In particular, the device is programmed such as to operate in accordance with a predetermined measurement schedule in addition to a prestored, guideline based schedule. In particular, a measurement schedule for making measurements during the day and during the night may be programmed together with the morning and evening measurements. These measurements lead to morning blood pressure results M BP, daytime measurement results D BP, evening measurment results E BP and night measurement results N BP. Again, these measurement results can be stored and/or used for calculation in the home measurement device.

Typically, two measurements may be made on each day in the morning and in the evening, typically between 6.00 am and 9.00 am and between 6.00 pm and 9.00 pm. These measurements may be triggered manually.

Measurements at work site and during the night may be triggered automatically, e.g. on one, two or three working days before next visit and with predetermined repetition frequency of 5-30 minutes during day and for 45 minutes during night time. The measurement parameters such as the measuring period and the interval time can be programmed in the home blood pressure measuring device 31 through the connection shown in FIG. 6.

In a second follow-up visit, a third blood pressure measurement is made at the point of care with the point of care measuring device leading to a blood pressure value CBP3. The time for the second follow-up visit is defined by the care person, e.g. by the physician. Typically, the second follow-up visit may be after two weeks. The patient is asked to individually measure blood pressure with the device 31 for e.g. 7 working days. This will lead to 12 readings which are required for reliable results. When the device is programmed in the “ABPM lite mode”, it is programmed such as to function only on specific days, e.g. before 1, 2 or 3 days.

An average CBP23, is formed on the basis of the second blood pressure measurements CBP2 and the third (second follow-up) blood pressure measurement CBP3. The blood pressure data acquired by self-measurement are subsequently downloaded to this system, in particular to a web based database.

The following data are determined on the basis of the data stored during home measurements in the home measurement device: an average value awakeBP is formed as the average of all morning, daytime and evening blood pressure measurements, an average asleepBP is formed as an average of all night time measurements,
an average workBP is formed as the average of all daytime measurements dayBP.
a morning average M_av is formed as the average of all morning measurements MBP,
an evening average E_av is formed as the average of all evening measurements E_BP.

A morning evening difference ME_diff is formed as the difference between the morning average and the evening average.

On the basis of these values, a masked hypertension and white coat hypertension measuring loop A is started (see FIG. 7b).

In a first step, it is judged whether the patient has white coat hypertension. If the awake blood pressure (awakeBP) is below 130/80 mmHG, it is judged that the patient suffered from white coat hypertension and that consequently there is a low risk. If the patient has no risk factors such as diabetes, target organ damage or other risk factors, lifestyle measures such as self blood pressure monitoring or other follow-up steps may be prescribed. No further measures may be necessary.

If the patient has other risk factors, reference is made to treatment guidelines and appropriate treatment is started.

If it is judged that no white coat hypertension is present for a patient, in a next step it is judged whether the patient suffers from masked hypertension. In an optional loop B (see FIG. 7c) presence and the kind of masked type hypertension can be detected.

In a first step, a parameter Night_reduction is formed as (awakeBP−asleepBP)/(awakeBP). If the parameter Night_reduction is <0.1 it is judged that the patient suffers from nocturnal hypertension. In a second step, it is judged whether the morning evening_average ME_av is above 135 and whether the morning evening difference ME_diff is above 20. If the answer is Yes in both cases, it is judged that the patient suffers from morning hypertension. In the next step, it is verified whether the average of work measurements workBP is above the morning evening averages ME_av. If the answer is Yes, it is judged that the patient suffers from daytime stress hypertension. The three types of masked hypertension are not independent from each other. Nocturnal hypertension typically presents the highest risk for the patient. The classification of the type of masked hypertension can help a care person to individually treat each patient. Patients suffering from nocturnal hypertension can be specifically prescribed with drugs to be taken before sleep.

In the next step the system displays or indicates which type of masked hypertension the patient is suffering from. This display is typically made on the system e.g. on an internet browser at the doctor's office.

In the next step (see FIG. 7b), it is analysed whether the blood pressure is dipping during the night. If the parameter Night_reduction is <0.1 it is judged that the pressure is not dipping and that the patient is at high risk.

If the blood pressure is dipping, in a next step it is judged whether the pulse pressure is <53 mmHG for the morning, evening, day and night measurements M_BP, E_BP, D_BP and N_BP.

The pulse pressure PP is formed as the average of all differences between the systolic and the diastolic blood pressure measurement (PP=average of all (Systolic BP−Diastolic BP)).

This value is calculated by the system as a temporary value on the basis of stored blood pressure values in view of pulse pressure judgement in FIG. 7b.

If the pulse pressure is below 53 mmHG, e.g. judged to be normal, the patient is at intermediate risk and is referred to specific guidelines for treatment. In particular, it is judged whether a drug treatment is necessary and depending on the answer, lifestyle measures are prescribed or a drug treatment is started.

If the pulse pressure is above 53 mmHG, it is judged that the patient is at high risk. A drug treatment is commenced in case the patient is found to be at high risk.

During the first visit, the patient profile is created. Blood pressure is measured on both arms and laboratory tests and home blood pressure measurement devices are prescribed.

During the second visit, laboratory test results are reviewed.

Upon the third visit, medication is prescribed and a plan of care is created. Prescription of medication and creation of a plan of care terminates the diagnosis phase.

During a further visit in the treatment phase, medication can be adjusted and a plan of care can be created or adjusted. During this visit, home measured blood pressure data are uploaded.

If necessary, a doctor can also prescribe the measurement schedule for morning, evening, night, and worksite measurements on the first visit. E.g. a patient having a normal office blood pressure on the first visit, still complaining about discomfortable symptoms during daytime or sleeping time can be prescribed with a measuring device based on a specific measurement schedule, e.g. including the ABPM lite function as described above with respect to FIG. 7a.physician.

FIG. 8 schematically shows the different steps carried out in accordance with the present invention. In the initial diagnosis phase, the individual is referred to a physician's office or more generally a point of care (physician's office) and the home blood pressure measurements are made during daytime and during the night as well as at the patient's home and at the worksite.

Drawbacks of traditional BP measurement methods such as white coat or masked hypertension, false reporting, human errors or blood pressure fluctuations are overcome. The system is non-expensive and thus overcomes one further drawback of traditional BP measurement methods.

If a patient is found to suffer from hypertension, a specific care plan with medication prescriptions and lifestyle changes in accordance with guidelines is established in a treatment phase. Medication is adjusted in an evidence based manner as will be shown hereinafter. In a follow-up phase, blood pressure values are monitored at home. Furthermore, compliance with medication also is monitored with the system in accordance with the present invention during this follow-up phase.

The commencement phase, treatment phase and follow-up phase together allow for a systematic and comprehensive approach to hypertension management and improvement. The pill box as shown in FIG. 3 reminds the patient to take drugs. Furthermore, intake history is registered in view of verification of medication compliance. The compliance data are sent to the web based database server from home or also from a pharmacy or a physician's office. With the assistance of medication and the blood pressure data, care persons can monitor the medication effect instantly. This systematic tool can help a physician to decide on the most suitable medication.

A somewhat alternative embodiment of a patient flow and care process in accordance with the invention is shown schematically in FIG. 9. FIG. 9 summarises another possibility for a flow chart based on the use of the system in accordance with the present invention. In parallel to home blood pressure measurements, laboratory tests may be prescribed. Furthermore, after a diagnosis, specific care plans and prescriptions may be handed out online by electronic prescription. Also, lifestyle changes may be prescribed. Furthermore, medication or nutraceuticals can be programmed on the basis of measurements and diagnosis made with the system of the present invention. By means of electronic communications and through telemedicine, the physician may communicate with the patients at home in view of further implementation of self health management. Furthermore, home blood pressure measurement and medication compliance data may be exchanged with a data base so as to be accessible through the physicians office.

The system may be further enhanced by care givers appointed to inspire the patient or to remind patient to follow-up. Also community health care organisations such as learning centres including dieticians, psychologists, therapists or pharmacists can be a part of the system by having access to the data gathered and stored by the system.

This system in accordance with the present invention includes a point of care blood pressure measuring device, a home blood pressure measuring device and a system for integrated management of measurements and measurements results provided by these devices. This system is based on an internet browser which allows display of data and entry of data in a web based database such as a standard Microsoft SQL server 2000.

In FIG. 10a, a browser page for entry of a patient profile for a specific patient is shown. In addition to personal data of the patient, specific treatment guidelines such as JNC7 (JNC7: the Seventh Report of the Joint National Committee on Prevention, Detection, Evaluation, and Treatment of High Blood Pressure), risk factors such as smoking, obesity or family history, associated clinical conditions or the weight are given. In addition, a home blood pressure measuring device prescribed to the patient is identified as a “tool”.

Operation of this tool is set up in a home BP management page shown in FIG. 10b. With the set up tool shown in FIG. 10b, measurement schedules carried out after the first follow-up visit (see FIG. 7a) are programmed in accordance with the steps shown in FIG. 7a. With the set up tool, a specific model of the device with an unambiguous serial number is defined. An ambulatory blood pressure measuring period is defined for a specific number of working days. A specific measurement schedule is defined for the morning and afternoon at worksite and during night time.

By saving the data shown in FIG. 10b, these data are stored in the database and can be transferred to the home blood pressure measuring device through the internet and USB connection as shown in FIG. 6.

FIG. 10c shows a screen of the patient data during the diagnosis phase. The diagnosis phase is defined as the phase of the initial, first and second follow-up visit as shown in FIG. 7a. On one and the same webpage, measurement results made with the point of care/office blood pressure measurement device and measurements made are with the home blood pressure measuring device before the first follow-up visit as well as measurements made with the ABPM Light device made between the first and second follow-up measurement are shown. The ABPM lite device and the device used for average home blood pressure measurement is physically the same device. The morning/evening function is a function embedded into the device. If the device is programmed in the ABPM lite mode, prescribed morning and evening measurements can be made by device. The ABPM lite function of the device can be activated or inactivated by the physician by means of a software switch. The ABPM lite function may be active during any of the diagnosis, treatment or follow-up phase at the physician's option.

Depending on whether or not a physician deems a morning or evening measurement appropriate, this function can be activated.

In addition laboratory tests, a specific plan of care (see also FIG. 11) and specific medication prescribed can be stored. After the end of the diagnosis phase, the treatment is started. By selecting a phase change checkbox with the browser based interface, the home blood pressure measuring device may be changed into a treatment mode. FIG. 10d shows a screen shot of the record of a specific patient during the treatment mode.

Blood pressure data are shown as a graph on the screen. In addition, medication prescribed and compliance with this medication is indicated. By direct correlation of medication prescribed and blood pressure monitors, a tool for evidence based medication adjustment is provided. A First type of medication is e.g. prescribed from May 5, 2006 to May 20, 2006. On May 20, the medication prescribed is changed. By respective colours in the horizontal axis in the graph, different kinds of medications are associated with the measurement results of the blood pressure measurements.

In addition, during the treatment phase, relevant measurements results are indicated. Details can be accessed easily by hyperlinks leading to more detailed measurement results.

Once the treatment is completed, the home blood pressure measuring device is changed into the follow-up mode. FIG. 10e shows a screen shot of the web browser for the specific patient shown in the previous figures in the follow-up mode. Several reminders for the physician such as reminders to make appointments, make specific tests for a case manager such as to upload home blood pressure data or for the patient such as medication related questions may be stored. These reminders may be sent to the respective person in a message box associated to each of the persons in this system. In the follow-up mode compliance information in association with blood pressure measurement results is indicated.

A specific embodiment of scenario including steps in a diagnosis phase, a treatment phase and a follow-up phase is shown in the following table:

| | | |
|---|---|---|
| Diagnosis | April 1, 1^{st} | Create patient profile |
| Phase | visit | Measure Office BP on both arms using WatchBP PRO on the initial visit mode |
| | | Prescribe lab test |
| | | Prescribe WatchBP Home for patient |
| | | Enable 1~3 days for measurement with ‘ABPM lite’ function if doctor think it is necessary, |
| | | also ask patient to measure at night and worksite on setup date. |
| | April 15, 2^{nd} | Measure Office BP on the arm with higher BP using the WatchBP PRO on the follow-up |
| | visit | visit mode |
| | | Upload WatchBP Home and ABPM lite data |
| | | View lab test result |
| | | Diagnosis follow the guideline |
| | | Prescribe medication |
| | | Create Plan of Care |
| | | Assign the education material for patient |
| Treatment | May 10, 3rd | Upload WatchBP Home and ABPM lite (if necessary) data at home and doctor view the |
| Phase | visit, through | home BP data and medication compliance information through WatchBP.NET |
| | on like | Doctor decide to adjust medication on May 10 |
| | internet | Doctor inform patient the change of medication by oral or by internet mail box. |
| | consultation | |
| | May 20, 4th | Measure Office BP on the arm with higher BP using the WatchBP PRO on the follow-up |
| | visit | visit mod |
| | | Upload WatchBP Home and ABPM lite (if necessary) data and medication compliance |
| | | result at office |
| | | Modify Plan of Care on May 20. |
| | | Doctor finishes medication adjustment if patient's BP has been controlled. |
| | | After May 20, patient takes medication and measure/upload BP at home. |

| | |
|---|---|
| Follow-up | After May 20, patient enters Follow-up Phase and takes medication and measure/upload BP at home and |
| Phase | the care manager in the call center can follow up the situation of the patient, |
| | e.g. (1) Nov. 5, Doctor views patients BP data through WatchBP.net and Informs patient the |
| | Time for next appointment on Nov. 20. |
| | (2) June 6, Doctor informs patient to take another blood test. |
| | (3) July 30, Doctor can setup WatchBP home and ABPM lite via internet connection and |
| | remind patient to take Home-BP with diagnostic mode for Aug 1~Aug 19 |
| | (4) Aug 1, Doctor reminds patient to take Home-BP with ABPM lite on Aug 2 |
| | (5) The care manager reminds patient to upload home-BP and ABPM lite date via |
| | internet connection. |
| | (6) Patient informs pharmacist for re-fill medication on July 20. |
| | (7) Patient consults pharmacist for medication-related question on July 10. |

In view of a plan of care for changing the patient's lifestyle, specific educational material may be assigned to the patient during the treatment phase or thereafter. A specific page for assigning material and for showing the material which has been assigned is shown in FIG. 10f. These information can be downloaded online.

During the first visit in the diagnosis phase, a patient profile is created. With the professional blood pressure measuring device “WatchBP Pro” the blood pressure is measured on both arms. Laboratory tests may be prescribed. Furthermore, the “WatchBP home” measuring device may be prescribed for home measurements by the patient. The WatchBP home measuring device be also programmed for 1, 2, or 3 ABPM lite measurements if this is deemed to be appropriate by the physician. The patient will then be asked to make night and worksite measurements.

During a second visit, the office blood pressure is measured by the physician on the arm having the higher blood pressure reading on the initial visit. The measurement data acquired by the patient at home are uploaded to the central data base. After a review of laboratory test results, a specific diagnosis may be made, medication may be prescribed and a plan of care can be created. In addition, optional educational material can be assigned to the patient online.

Afterwards, a treatment phase is started. A third visit can be made online, e.g. through internet consultation. Home measurement blood pressure data from the “WatchBP home” or “ABPM lite” device (if prescribed) can be uploaded. A care person such as a physician can review these data and also medication compliance through the browser interface, called hereinafter “WatchBPnet”. Necessary medication adjustments may be made and the patient may be informed about the change of medication orally or through e-mails integrated in the system.

During a fourth visit, the office blood pressure again is measured on the arm having the higher initial blood pressure reading with the office measurement device.

The home acquired data and medication compliance data are uploaded to the physician's office or to the central data base. A plan of care can be modified and medication adjustments can be finalised. After this meeting, further medication is taken at home and home measurements are made.

Thereafter, the follow-up phase is started. A care manager in a call centre can individually follow a patient. Followup can e.g. include review of patient's blood pressure data through the browser interface. Physicians also can make further appointments. Physicians can inform patients to make further laboratory tests. It is also possible for a physician to set up the function of the home device through an internet connection and to remind the patient to take further measurements in a diagnostic mode for a predetermined period of time. Furthermore, communication with care managers or pharmacists in view of uploading data, refilling medication or compliance with medication is possible.

FIG. 11 schematically shows a plan of care for a specific patient. Relevant measurement data are shown and the specific plan and drugs prescribed for the patient are shown.

FIG. 12 schematically shows an example of a pay for performance report made by a specific physician for a plurality of patients. The system in accordance with the present invention provides for generation of pay for performance reports. FIG. 12 shows the report of a specific physician including measurement results made for several patients.

P4P reports, assignment of material, messages in the message box or patient related information may be selected by selection of appropriate tabs in the first line of the pages shown in FIGS. 10c to 10f.

FIG. 13 shows a personal medication record including all the medication and alternative prescriptions made. Furthermore, a summary of all laboratory and other measurements are shown. This personal medication record is easily accessible to the physician starting from a management page (see below).

FIGS. 14a to 14j show different screen shots of a medication therapy management (MTM) functionality.

FIG. 14a shows a patient management page. The care persons such as the physician or pharmacist may select a medication therapy management mode for the patients listed (by clicking on an MTM icon for each patient). By clicking this icon, a further page for the selected patient will be opened showing a plurality of entries for the patient, including the date of modification of data as well as actual times spent by the physician or pharmacist for the patient (see FIG. 14b. By selecting an edit icon, the data for the specific patient may be amended. FIG. 14c shows a page for editing patient data. General patient data, names of a pharmacist and the primary physician and specific types of medicaments, herbs, supplements, associated adverse reactions or side effects can be entered by simply clicking on icons shown in the last column in FIG. 14a. Furthermore, laboratory and biological data may be entered and reviewed.

By clicking on the appropriate icon directed to medication, specific medication can be selected by means of a general category and a specific type of medication. Furthermore, medication can be administered, e.g. by means of number of takings per day, dosages, times of drug intake or numbers of days for drug intake. Adverse reactions may be selected on the basis of a predefined list shown in FIG. 14c. Furthermore, individual adverse reactions may be entered.

In the screen shot shown in FIG. 14d, specific actions can be listed and attributed to specific care persons. By further scrolling down (see FIG. 14g) further information such as blood pressure values or physical measurements, related issues, proposed actions, received education or results of action as well as intervention measures/referrals can be accessed. Related issues e.g. can be entered or amended by clicking an appropriate icon (see FIG. 14h). Also, proposed actions may be entered or amended by clicking appropriate icons (see FIG. 14i). Once the data have been entered, the data can be directly seen in the overall page (see FIG. 14j).

By selecting the print icon in FIG. 14f, a personal medication record as shown in FIG. 13 can be generated. Different types of reports can be selected on demand and exported as PDF, text or spread sheet files.

1-30. (canceled)

31. A system for hypertension management, the system comprising
at least one home blood pressure measurement device for blood pressure measurements at an individual's home
at least one point of care measuring system with a point of care blood pressure measuring device for blood pressure measurement at a point of care,
wherein the system is provided with data communication means for transferring data from said home blood pressure measuring device to a central data base and/or said point of care measuring system.

32. A system according to claim 31, wherein at least one of said home blood pressure measuring device or said point of care blood pressure measuring device is adapted to determine blood pressure values in accordance with predetermined measurement guidelines.

33. A system according to claim 32, wherein said home blood pressure measuring device has an interface for entering a measurement schedule based on said measurement guidelines, in particular an interface formed by said data communication means.

34. A system according to claim 33, wherein said home blood pressure measuring device is operable in a first, automatic measurement mode and in a second, manual measurement mode,
wherein the measurement mode is preferably determined by the measurement schedule.

35. A system for hypertension management, wherein this system is provided with calculating means adapted for determination of at least one of white coat hypertension or masked hypertension on the basis of measurements made with a home blood pressure measuring devices.

36. A system according to claims 31, wherein the home blood pressure measuring devices and the hypertension management system and preferably the home blood pressure measuring device are operable in a diagnostic operating mode, a treatment operating mode and a follow-up operating mode and wherein the operating mode preferably can be set through an interface formed by said data communication means.

37. A system for hypertension management, comprising at least one point of care measuring device for blood pressure measurements at a point of care,
wherein said point of care blood pressure measuring device is designed to operate in accordance with predetermined quality standards, in particular in accordance with guidelines of a pay for performance program or in accordance with a medication therapy management program.

38. A system according to claim 31, the system further comprising at least one home based medication compliance monitoring device, said medication compliance monitoring device being designed for data communication with a central database.

39. A system according to claim 31, wherein the system is further provided with a centralised web based software tool and database accessible by at least one of the point of care measuring system and the home blood pressure measuring system.

40. A system according to claim 39, wherein the web based database includes a disease management knowledge database accessible by the individual.

41. A system according to claim 39, wherein the web based database includes tools for physicians in view of providing treatments based on guidelines.

42. A system according to claim 39, wherein the web based database includes tools for pharmacists to ensure drug compliance.

43. A system in accordance with claim 39, comprising web based software tools in view of generation of reports in accordance with predetermined quality standards.

44. A system according to claim 31, wherein the system includes at least one point of care measuring system at a physician's office and at least one point of care measuring system at a pharmacy.

45. A system according to claim 31, the system further comprising means for generating individual education reports for said individual.

46. A blood pressure measuring device for blood pressure measurements of an individual at a point of care, wherein the blood pressure measurement device is provided with means for generating reports to be used in a quality insurance program, in particular in a pay for performance or a medication therapy management program.

47. A method for hypertension management, the method comprising the steps of
determining blood pressure values of an individual at the individual's home with a home blood pressure measuring device
determining blood pressure values of said individual at a point of care with a point of care blood pressure measuring device
transferring data from said home blood pressure measuring device to a point of care blood pressure measuring system and/or a central database.

48. A method according to claim 47, wherein determination of blood pressure values of the individual at the individual's home is made in accordance with predetermined measurement criteria.

49. A method according to claim 48, wherein a measurement schedule based on said measurement guidelines is entered into the home blood pressure measuring device through an interface, in particular by data communication means for transferring data from the home blood pressure measuring device to a point of care or to the central database or by means of a direct connection of the device to a computer.

50. A method according to claim 47, comprising the further step of determining whether a patient suffers from masked hypertension, nocturnal hypertension or white coat hypertension, in particular on the basis of values determined at the individual's home and at physician's office.

51. A method according to claim 47, wherein determination of blood pressure values at the point of care is made in accordance with predetermined measurement criteria.

52. A method for hypertension management, comprising the steps of
determining blood pressure values of an individual at a point of care with a point of care blood pressure measuring device
automatically generating reports for a quality standard program, in particular reports for a pay for performance program or for a medication therapy management program.

53. A method according to claim 47, comprising the further step of gathering home based medication compliance information with a home based medication compliance monitoring device and transmitting said compliance data to a central database.

54. A method according to claim 47, comprising the further steps of generating education reports for said individual based on information contained in a data base of said management system.

55. A method according to claim 47, wherein the individual accesses a disease management knowledge database arranged on a central web database.

56. A method according to claim 47, wherein blood pressure data determined by the individual at home with a home blood pressure measurement device and blood pressure data determined at the point of care with the point of care blood pressure measuring device are transmitted to a central database, preferably a web based database.

57. A system according to claim 31, wherein the system comprises a central database including at least the following data:
patient related data
home blood pressure management data
point of care management results
home measurement results
optionally, laboratory test results
optionally, plan of care elements
optionally, medication information
optionally, office physical measurements
optionally, assigned material
optionally, P4P reports
optionally, personally medication records.

58. A system according to claim 31, wherein the home blood pressure measurement device is operable in at least two home measurement modes, wherein in a normal measurement mode, measurements can be manually made in the morning and in the evening according to a prestored measurement schedule and wherein in an extended measurement mode, measurements are additionally made, preferably automatically, during daytime and/or during the night in accordance with a measurement schedule stored in the device.

59. A system according to claim 58, wherein the system comprises a switch, preferably a software implemented switch, for switching the device between the normal measurement mode and the extended measurement mode.
